(19) United States
(12) Patent Application Publication
(10) Pub. No.: US 2024/0188896 A1
(43) Pub. Date: Jun. 13, 2024

(54) DYNAMIC STRESS SCORING WITH PROBABILITY DISTRIBUTIONS

(71) Applicant: Whoop, Inc., Boston, MA (US)

(72) Inventors: Laura Ware, Boston, MA (US); Emily Rachel Capodilupo, Boston, MA (US)

(21) Appl. No.: 18/536,730

(22) Filed: Dec. 12, 2023

Related U.S. Application Data
(60) Provisional application No. 63/594,258, filed on Oct. 30, 2023.
(60) Provisional application No. 63/431,877, filed on Dec. 12, 2022.

(51) Int. Cl. A61B 5/00; A61B 5/0205; A61B 5/16

(52) U.S. Cl. CPC A61B 5/7267; A61B 5/0205; A61B 5/165; A61B 5/486; A61B 5/7246; A61B 5/742; A61B 5/1118

(57) ABSTRACT

A stress score from a physiological monitor provides a local, objective, quantitative measurement of stress in a numerical form that can be used as the basis for real time coaching, decision-making, and so forth.

### CROSS-REFERENCE TO RELATED APPLICATIONS

This application claims priority to U.S. Provisional Patent App. No. 63/594,258 filed on Oct. 30, 2023, and U.S. Provisional Patent App. No. 63/431,877 filed on Dec. 12, 2022, where the entire contents of each of the foregoing applications are hereby incorporated by reference herein.

### TECHNICAL FIELD

The present disclosure generally relates to generating a stress score based on data from a wearable physiological monitor.

### BACKGROUND

Some physiological monitoring devices acquire user data over an interval, such as several days, and then generate a daily summary of metrics for, e.g., sleep, strain, and recovery. These calculations are frequently data heavy and computationally expensive, requiring, as they do, a review of accumulated data over the course of a prior period such as twenty four hours.

There remains a need for continuous or real time objective measurements of current stress that provides an actionable benchmark for user decisions and self-assessment.

### SUMMARY

A stress score from a physiological monitor provides a local, objective, quantitative measurement of stress in a numerical form that can be used as the basis for real time coaching, decision-making, and so forth.

In some aspects, a method disclosed herein may include: providing a heart rate variability metric for a user; providing a heart rate metric for the user; determining a resting state of the user; and calculating a stress score for the user where the stress score is calculated based on a weighted combination of the heart rate variability metric and the heart rate metric, the weighted combination uses a first weight for a first component of the stress score based on the heart rate metric, where the first weight is based on the resting state of the user, and the weighted combination uses a second weight for a second component of the stress score based on the heart rate variability metric, where the second weight is based on the resting state of the user. Other embodiments of this aspect include corresponding computer systems, apparatus, and computer programs recorded on one or more computer storage devices, each configured to perform the actions of the methods.

Implementations may include one or more of the following features. The second weight may be equal to one minus the first weight. The first weight may approach 1 as the heart rate metric approaches a resting heart rate for the user. The first weight may monotonically approach 1 as the heart rate metric approaches the resting heart rate. The first weight may sigmoidally approach 1 as the heart rate metric approaches the resting heart rate. The first weight may increase according to a proximity of the resting state to a sleep state for the user. The method may include periodically calculating the stress score over an interval, thereby providing a timeline of stress scores for the user over the interval. The method may include displaying the timeline of stress scores on a user device as a stress score graph. Providing the heart rate variability metric and the heart rate metric may include acquiring the heart rate variability metric and the heart rate metric from a physiological monitor worn by the user. The method may include displaying the stress score on one or more of a wearable physiological monitor and a user device. The method may include generating an intervention recommendation for the user based on the stress score. The intervention recommendation may include a real time recommendation based on a current stress score. The intervention recommendation may include a real time recommendation based on a current activity. The method may include identifying a threshold for the stress score that is indicative of acute stress. The method may include reporting the acute stress to the user. The method may include recommending a remediation for the acute stress to the user. The method may include identifying a threshold for the stress score that is indicative of autonomic activation. The resting state may be based on a circadian state of the user. The circadian state may be based on a probability of a sleep state for the user. The circadian state may be based on a circadian rhythm model for a population. The circadian state may be based on a circadian rhythm model for the user. The resting state may be based on a difference between the heart rate metric of the user and a resting heart rate of the user. The resting state may be a discrete resting state including at least one of an asleep state and an awake state. The asleep state may include one or more sub-states for one or more corresponding stages of sleep. The first weight for weighting the heart rate metric may increase when the resting state is the asleep state. The resting state may be a discrete resting state selected based on one or more predetermined ranges for the heart rate. Providing the heart rate metric may include acquiring an aggregate heart rate measurement over an interval with a wearable physiological monitor. Providing the heart rate variability metric may include acquiring an aggregate heart rate variability measurement over the interval with the wearable physiological monitor. The method may include adjusting the stress score based on motion data obtained from a wearable monitor worn by the user. Providing the heart rate variability metric may include scaling a heart rate variability measurement based on a distribution of historical heart rate variability measurements. Providing the heart rate metric may include scaling a heart rate measurement based on a distribution of historical heart rate measurements. Implementations of the described techniques may include hardware, a method or process, or computer software on a computer-accessible medium.

In some aspects, a computer program product disclosed herein may include computer executable code embodied in a non-transitory computer readable medium that, when executing on one or more computing devices, causes the one or more computing devices to perform the steps of: providing a heart rate variability metric for a user; providing a heart rate metric for the user; determining a resting state of the user; and calculating a stress score for the user where—the stress score is calculated based on a weighted combination of the heart rate variability metric and the heart rate metric, the weighted combination uses a first weight for the heart rate metric based on the resting state of the user, and the weighted combination uses a second weight for the heart rate variability metric based on the resting state of the user.

In some aspects, a system disclosed herein may include a wearable physiological monitor including one or more sensors and a first processor configured to continuously acquire heart rate data for a user based on a signal from the one or more sensors; and one or more processors coupled in a communicating relationship with the wearable physiological monitor. The one or more processors may be configured by computer executable code to receive data from the wearable physiological monitor and to: calculate a heart rate variability metric for the user based on the heart rate data; calculate a heart rate metric for the user based on the heart rate data; determine a resting state of the user based on the heart rate data; and calculate a stress score for the user where—the stress score is calculated based on a weighted combination of the heart rate variability metric and the heart rate metric, the weighted combination uses a first weight for the heart rate metric based on the resting state of the user, and the weighted combination uses a second weight for the heart rate variability metric based on the resting state of the user. The system may also include a display device in communication with the one or more processors, the display device including a user interface configured to present a value to the user indicative of the stress score.

Implementations may include one or more of the following features. At least one processor of the one or more processors may be disposed on the display device. At least one processor of the one or more processors may be disposed on a remote server. At least one processor of the one or more processors may be disposed on a user device. A processor of the one or more processors may be disposed on a remote server, and another processor of the one or more processors may be disposed on a user device.

In some aspects, a computer program product disclosed herein may include computer executable code embodied in a non-transitory computer readable medium that, when executing on one or more computing devices, performs the steps of: providing a machine learning model trained to report a stress level based on a heart rate, a heart rate variability, and a motion measured from a monitor; acquiring a plurality of measurements of the stress level based on data from the monitor for a user over an interval; processing the plurality of measurements of the stress level over the interval to provide a stress estimate for the interval; scaling the stress estimate with a function that transforms the stress estimate into a value within a predetermined range; and presenting the value to the user in a display as a dynamic stress value.

In some aspects, a method disclosed herein may include: providing a machine learning model trained to report a stress level based on a heart rate, a heart rate variability, and a motion measured from a monitor; acquiring a plurality of measurements of the stress level based on data from the monitor for a user over an interval; and processing the plurality of measurements of the stress level over the interval to provide a stress estimate for the interval. Other embodiments of this aspect include corresponding computer systems, apparatus, and computer programs recorded on one or more computer storage devices, each configured to perform the actions of the methods.

Implementations may include one or more of the following features. The method may include scaling the stress estimate with a function that transforms the stress estimate into a value within a predetermined range, and presenting the value to the user in a display as a dynamic stress value. The method may include displaying the dynamic stress value on a wearable monitor. The method may include displaying the dynamic stress value on a user device. The function may include a non-linear scaling that transforms a majority of a number of stress estimates for an individual to a lowest value for the dynamic stress value. The method may include generating an intervention recommendation for the user based on the stress estimate. The intervention recommendation may include a real time recommendation based on a current stress estimate. The intervention recommendation may include a real time recommendation based on a current activity. The machine learning model may be trained using a training set that includes a set of measured physiological responses to one or more predetermined stressors for a plurality of users, each tagged with a stress score from a corresponding one of the plurality of users when exposed to a corresponding one of the one or more predetermined stressors. The method may include identifying a threshold for the stress estimate that is indicative of acute stress. The method may include reporting the acute stress to the user. The method may include recommending a remediation for the acute stress to the user. The method may include identifying a threshold for the stress estimate that is indicative of autonomic activation. The plurality of measurements of the stress level may include measurements at least every thirty seconds. The interval may be between three minutes and ten minutes. Implementations of the described techniques may include hardware, a method or process, or computer software on a computer-accessible medium.

In some aspects, a method disclosed herein may include: providing a model configured to output a stress level based on a heart rate, a heart rate variability, and a motion measured from a monitor; acquiring a plurality of measurements of the stress level based on data from the monitor for a user over an interval; and processing, using the model, the plurality of measurements of the stress level over the interval to provide a stress estimate for the interval. Other embodiments of this aspect include corresponding computer systems, apparatus, and computer programs recorded on one or more computer storage devices, each configured to perform the actions of the methods.

Implementations may include one or more of the following features. The model may include a machine learning model trained to report the stress level based on the heart rate, the heart rate variability, and the motion measured from the monitor. The model may include an analytical model using a combination of a scaled heart rate score and a scaled heart rate variability score. The analytical model may weight a contribution of the scaled heart rate score and the scaled heart rate variability score based on motion detected by the monitor. Implementations of the described techniques may include hardware, a method or process, or computer software on a computer-accessible medium.

In some aspects, a computer program product disclosed herein may include computer executable code embodied in a non-transitory computer readable medium that, when executing on one or more computing devices, performs the steps of: creating a first model, the first model including a machine learning model trained to generate a probability distribution of expected heart rate reserve ratios based on a first set of features of training data for a population of users of a type of physiological monitor; receiving user data from a wearer of a first physiological monitor of the type of physiological monitor; calculating a heart rate reserve ratio for the wearer based on the user data from the first physiological monitor; generating the probability distribution of expected heart rate reserve ratios for the wearer based on the first set of features of the user data; and calculating a stress score for the wearer based on a comparison of the heart rate reserve ratio to the probability distribution of expected heart rate reserve ratios.

Implementations may include one or more of the following features. The computer program product may include code that performs the step of creating a second model, the second model including a classification model trained to identify an activity type based on a second set of features of the training data for the population of users of the type of physiological monitor. The second model may be used to modify the stress score to better match an expected stress score of the wearer. The activity type may include one or more of active, sedentary, and sleeping. The computer program product may include code that performs the steps of classifying an activity type of a user based on a second set of features of the user data and refining the stress score based on the activity type, thereby providing a refined stress score. The computer program product may include code that performs the step of providing recommendations to the user based on the refined stress score. The activity type may include one or more of active, sedentary, and sleeping. Generating the probability distribution may include generating a set of heart rate reserve ratios corresponding to each of a number of quantiles for the probability distribution. The computer program product may include code that performs the step of displaying the stress score on one or more of a wearable monitor and a user device. The computer program product may include code that performs the step of generating an intervention recommendation for the wearer based on the stress score. The intervention recommendation may include a real time recommendation based on a current stress score. The intervention recommendation may include a real time recommendation based on a current activity. The computer program product may include code that performs the step of identifying a threshold for the stress score that is indicative of acute stress. The computer program product may include code that performs the step of reporting the acute stress to the wearer. The computer program product may include code that performs the step of recommending a remediation for the acute stress to the wearer. The computer program product may include code that performs the step of identifying a threshold for the stress score that is indicative of autonomic activation. Implementations of the described techniques may include hardware, a method or process, or computer software on a computer-accessible medium.

In some aspects, a method disclosed herein may include: creating a first model, the first model including a machine learning model trained to generate a probability distribution for a physiological metric based on a first set of features of training data for a population of users of a type of physiological monitor; receiving user data from a wearer of a first physiological monitor of the type of physiological monitor; calculating a value for the physiological metric for the wearer based on the user data from the first physiological monitor; generating the probability distribution for the physiological metric for the wearer based on the first set of features of the user data; and calculating a stress score for the wearer based on a comparison of the value for the physiological metric to the probability distribution for the physiological metric. Other embodiments of this aspect include corresponding computer systems, apparatus, and computer programs recorded on one or more computer storage devices, each configured to perform the actions of the methods.

Implementations may include one or more of the following features. The method may include creating a second model, the second model including a classification model trained to identify an activity type based on a second set of features of the training data for the population of users of the type of physiological monitor. The second model may be used to modify the stress score to better match an expected stress score of the wearer. The physiological metric may include a heart rate metric. The physiological metric may include a metric correlated to stress. The physiological metric may include one or more of a heart rate reserve, a heart rate reserve ratio, a heart rate variability, an instantaneous heart rate, an aggregate heart rate, a skin temperature, a core body temperature, a respiratory rate, blood pressure, and a skin conductance. Implementations of the described techniques may include hardware, a method or process, or computer software on a computer-accessible medium.

In some aspects, a method disclosed herein may include: measuring an aggregate heart rate of a user over an interval with a physiological monitor; determining whether the aggregate heart rate over the interval is within a predetermined range of a resting heart rate for the user; in response to determining that the aggregate heart rate is outside the predetermined range, calculating a stress score for the user over the interval based on a plurality of measurements of each of a heart rate, a heart rate variability, and a motion acquired during the interval from the physiological monitor; in response to determining that the aggregate heart rate is within the predetermined range, calculating the stress score for the user based on the plurality of measurements using a lower weighted contribution of the heart rate variability relative to the heart rate than a weighted contribution used when it is determined that the aggregate heart rate is outside the predetermined range; and displaying a value to the user indicative of the stress score for the interval. Other embodiments of this aspect include corresponding computer systems, apparatus, and computer programs recorded on one or more computer storage devices, each configured to perform the actions of the methods.

Implementations may include one or more of the following features. The method may include updating a cumulative stress score for the user based on the stress score. The method may include, in response to determining that the aggregate heart rate is outside the predetermined range, applying a first algorithm to calculate the stress score for the user, and, in response to determining that the aggregate heart rate is within the predetermined range, applying a second algorithm to calculate the stress score for the user, the second algorithm including the lower weighted contribution of the heart rate variability relative to the first algorithm. The lower weighted contribution of the heart rate variability may be scaled relative to a distance from the resting heart rate for the user. The distance may include a sigmoidal distance to resting heart rate determined using a sigmoid function that assesses proximity of the heart rate to the resting heart rate for the user. The stress score may be scaled with a function that transforms the stress score into the value. Scaling of the stress score places the value within a predetermined range. The value may be displayed on at least one of the physiological monitor and a user device in communication with the physiological monitor. The method may include generating an intervention recommendation for the user based on the stress score. Implementations of the described techniques may include hardware, a method or process, or computer software on a computer-accessible medium.

In some aspects, a computer program product disclosed herein may include computer executable code embodied in a non-transitory computer readable medium that, when executing on one or more computing devices, performs the steps of: measuring an aggregate heart rate of a user over an interval with a physiological monitor; determining whether the aggregate heart rate over the interval is within a predetermined range of a resting heart rate for the user; in response to determining that the aggregate heart rate is outside the predetermined range, calculating a stress score for the user over the interval based on a plurality of measurements of each of a heart rate, a heart rate variability, and a motion acquired during the interval from the physiological monitor; in response to determining that the aggregate heart rate is within the predetermined range, calculating the stress score for the user based on the plurality of measurements using a lower weighted contribution of the heart rate variability relative to the heart rate than a weighted contribution used when it is determined that the aggregate heart rate is outside the predetermined range; and displaying a value to the user indicative of the stress score for the interval.

In some aspects, a system disclosed herein may include: a wearable physiological monitor including one or more sensors and a first processor configured to continuously acquire data for a user based on a signal from the one or more sensors, the data including a heart rate, a heart rate variability, and a motion; and a second processor coupled in a communicating relationship with the wearable physiological monitor. The second processor may be configured by computer executable code to receive data from the wearable physiological monitor and to: measure an aggregate heart rate of the user over an interval; determine whether the aggregate heart rate over the interval is within a predetermined range of a resting heart rate for the user; in response to determining that the aggregate heart rate is outside the predetermined range, calculate a stress score for the user over the interval based on the data from the wearable physiological monitor acquired during the interval; and in response to determining that the aggregate heart rate is within the predetermined range, calculate the stress score for the user based on the data from the wearable physiological monitor acquired during the interval using a lower weighted contribution of the heart rate variability relative to the heart rate than a weighted contribution used when it is determined that the aggregate heart rate is outside the predetermined range. The system may also include a display device in communication with the second processor, the display device including a user interface configured to present a value to the user indicative of the stress score for the interval. The second processor may be disposed on one or more of the wearable physiological monitor, the display device, and a remote server.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 illustrates a physiological monitoring system.

FIG. 3 shows a smart garment system.

FIG. 4 is a block diagram of a computing device.

FIG. 5 shows a system for dynamic stress monitoring.

FIG. 6 is a flowchart of a method for dynamic stress monitoring.

FIG. 7 is a flowchart of a method for determining a stress score.

FIG. 8 illustrates a process for calculating a stress score.

FIG. 9 shows a user interface displaying a dynamic stress score.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night). More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component or module of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as, DRAM, SRAM, EDO RAM), and so forth. Although not depicted, any of the devices or components described herein may include a computer-readable medium or other memory for storing program instructions, data, and the like.

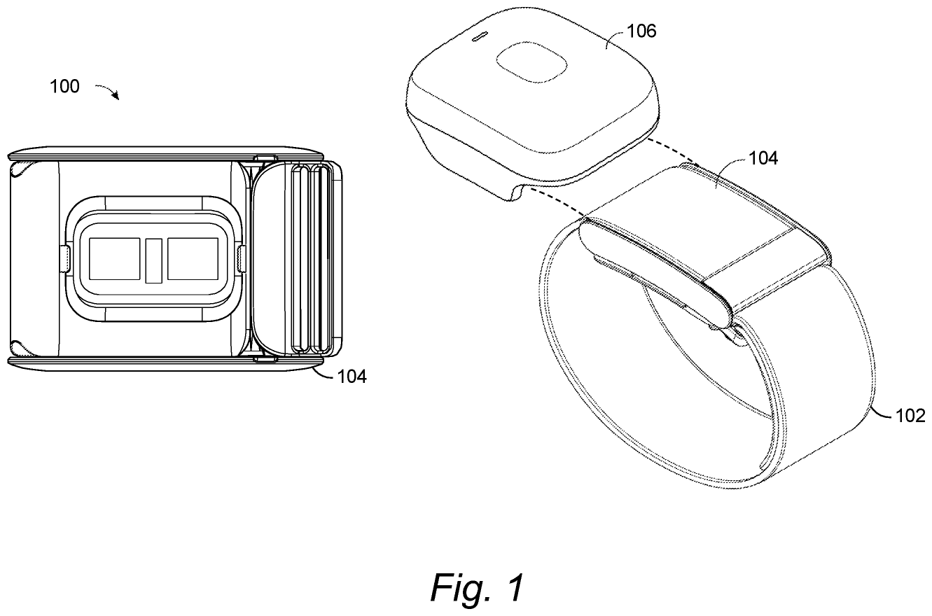

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, an ankle, a bicep, a chest, or any other suitable location(s), and the strap 102 may be, or may include, a waistband or other elastic band or the like within an article of clothing or accessory. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 describes non-limiting example embodiments of suitable wearable monitors 104, and is incorporated herein by reference in its entirety.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiogra any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by the wearer's skin.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure, and the like, as well environmental or contextual parameters such as ambient light, ambient temperature, humidity, time of day, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting addition functions of the wearable monitor 104. For example, the system 100 may include communications systems to support, e.g., near field communications, proximity sensing, Bluetooth communications, Wi-Fi communications, cellular communications, satellite communications, and so forth. The wearable monitor 104 may also or instead include components such as a geopositioning system (e.g., based on the Global Positioning System or GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. Also or instead, the system 100 may include a plurality of wearable monitors 104 (and/or other physiological monitors) that can share battery power or provide power to one another. The system 100 may perform numerous functions related to continuous monitoring, such as automatically detecting when the user is asleep, awake, exercising, and so forth, and such detections may be performed locally at the wearable monitor 104 or at a remote service coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis.

In one aspect, the wearable monitor may be a wrist-worn photoplethysmography device.

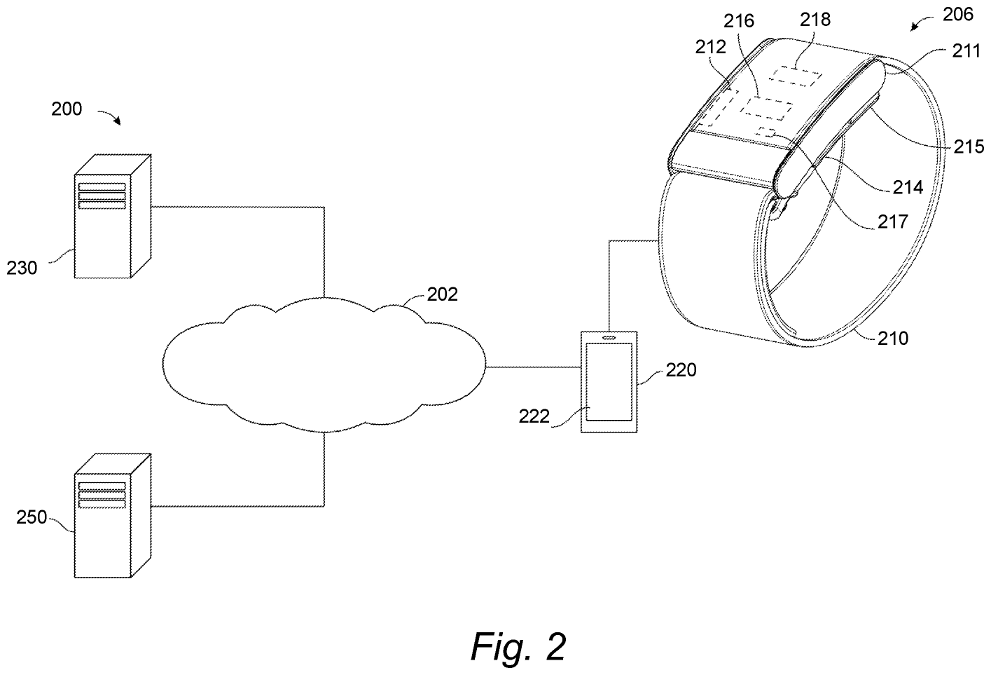

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communicating with local resources. By way of non-limiting examples, short range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206.

The one or more sensors 214 may include any of the sensors described herein, or any other sensors or sub-systems suitable for physiological monitoring or supporting functions. By way of example and not limitation, the one or more sensors 214 may include one or more of a light source, an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, Global Positioning System hardware/software, a proximity sensor, an RFID tag reader, and RFID tag, a temporal sensor, an electrodermal activity sensor, and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicated detected motion, and/or beacons or the like to self-identify type, weight, and so forth, in order to facilitate automated detection and tracking of exercise activity with other connected devices.

One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issues, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrocardiogram (ECG) based heart rate measurements to be gathered from a torso region of the wearer; wireless antennas to be placed above the upper portion of the thoracic spine to achieve desired communications signals; a contactless payment system to be embedded in a sleeve cuff for interactions with a payment terminal; and muscle oxygen saturation measurements to be gathered from muscles such as the pectoralis major, latissimus dorsi, biceps brachii, and other major muscle groups. This non-exhaustive list illustrates just some examples of technology that may be incorporated into a single garment.

Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

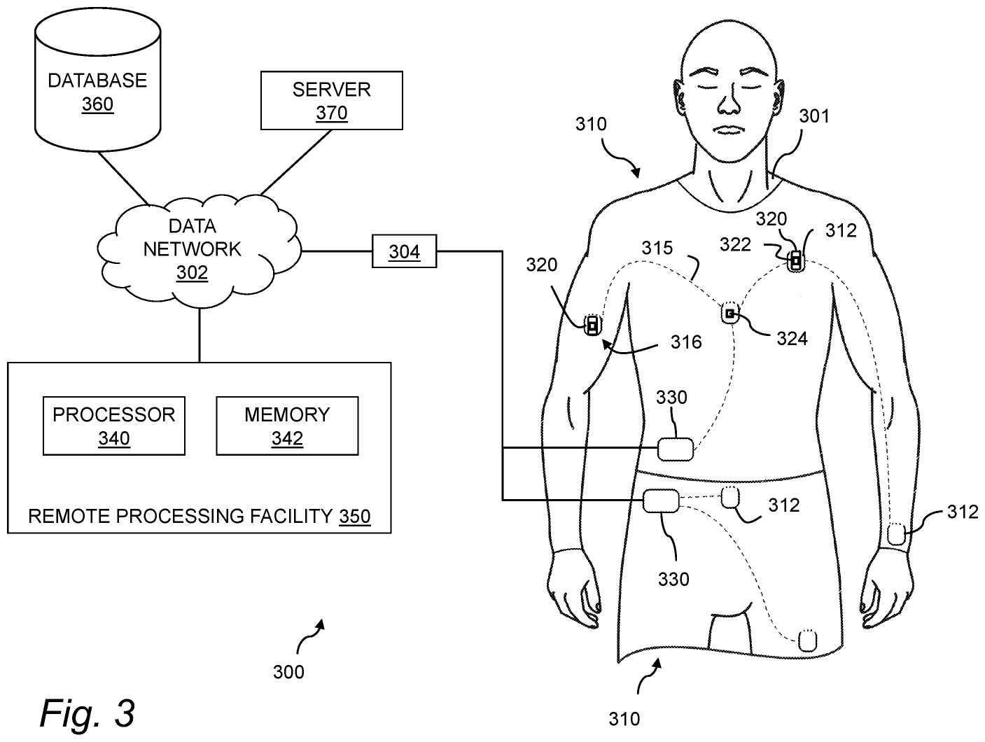

FIG. 3 shows a smart garment system. In general, the system 300 may include a plurality of components—e.g., a garment 310, one or more modules 320, a controller 330, a processor 340, a memory 342, and so on—capable of communicating with one another over a data network 302. The garment 310 may be wearable by a user 301 and configured to communicate with a module 320 having a physiological sensor 322 that is structurally configured to sense a physiological parameter of the user 301. As discussed herein, the module 320 may be controllable by the controller 330 based at least in part on a location 316 where the module 320 is located on or within the garment 310. This position-based information may be derived from an interaction and/or communication between the module 320 and the garment 310 using various techniques. It will be understood that, while two controllers 330 are shown, the garment 310 may include a single inter-garment controller, or any number of separate controllers 330 in any number of garments 310 (e.g., one per garment, or one for all garments worn by a person, etc.), and/or controllers may be integrated into other modules 320.

For communication over the data network 302, the system 300 may include a network interface 304, which may be integrated into the garment 310, included in the controller 330, or in some other module or component of the system 300, or some combination of these. The network interface 304 may generally include any combination of hardware and software configured to wirelessly communicate data to remote resources. For example, the network interface 304 may use a local connection to a laptop, smartphone, or the like that couples, in turn, to a wide area network for accessing, e.g., web-based or other network-accessible resources. The network interface 304 may also or instead be configured to couple to a local access point such as a router or wireless access point for connecting to the data network 302. In another aspect, the network interface 304 may be a cellular communications data connection for direct, wireless connection to a cellular network or the like.

The data network 302 may generally include any communication network through which computer systems may exchange data. For example, the data network 302 may include, but is not limited to, the Internet, an intranet, a LAN (Local Area Network), a WAN (Wide Area Network), a MAN (Metropolitan Area Network), a wireless network, a cellular data network, an optical network, and the like. To exchange data via the data network 302, the system 300 and the data network 302 may use various methods, protocols, and standards including, but not limited to, token ring, Ethernet, wireless Ethernet, Bluetooth, TCP/IP, UDP, HTTP, FTP, SNMP, SMS, MMS, SS7, JSON, XML, REST, SOAP, CORBA, IIOP, RMI, DCOM and Web Services. To ensure data transfer is secure, the system 300 may transmit data via the data network 302 using a variety of security measures including, but not limited to, TSL, SSL and VPN. By way of example, some embodiments of the system 300 may be configured to stream information wirelessly to a social network, a data center, a cloud service, and so forth.

In some embodiments, data streamed from the system 300 to the data network 302 may be accessed by the user 301 (or other users) via a website. The network interface 304 may thus be configured such that data collected by the system 300 is streamed wirelessly to a remote processing facility 350, database 360, and/or server 370 for processing and access by the user. In some embodiments, data may be transmitted automatically, without user interactions, for example by storing data locally and transmitting the data over available local area network resources when a local access point such as a wireless access point or a relay device (such as a laptop, tablet, or smartphone) is available. In some embodiments, the system 300 may include a cellular system or other hardware for independently accessing network resources from the garment 310 without requiring local network connectivity.

In one example, the network interface 304 may be configured to stream data using Bluetooth or Bluetooth Low Energy technology, e.g., to a nearby device such as a cell phone or tablet for forwarding to other resources on the data network 302. In another example, the network interface 304 may be configured to stream data using a cellular data service, such as via a 3G, 4G, or 5G cellular network. It will be understood that the network interface 304 may include a computing device such as a mobile phone or the like. The network interface 304 may also or instead include or be included on another component of the system 300, or some combination of these. Where battery power or communications resources can advantageously be conserved, the system 300 may preferentially use local networking resources when available, and reserve cellular communications for situations where a data storage capacity of the garment 310 is reaching capacity. Thus, for example, the garment 310 may store data locally up to some predetermined threshold for local data storage, below which data is transmitted over local networks when available. The garment 310 may also transmit data to a central resource using a cellular data network only when local storage of data exceeds the predetermined threshold.

The garment 310 may provide suitable compression to hold the module 320 against the wearer's skin with an appropriate level of force for signal acquisition and/or stability for one or more sensors included therein or thereon. The garment 310 may include one or more layers of fabric—e.g., multiple layers with an interior layer providing compression and an exterior layer providing an aesthetic or other function. The garment 310 may include one or more of a shirt (or other top), shorts/pants (or other bottom), an undergarment (e.g., undershirt, underwear, brassiere, and so on), a sock or other footwear, a shoe, a facemask, a hat or helmet (or other head adornment), a compression sleeve, a sweatband, kinesiology tape or elastic therapeutic tape, a glove, and the like. More generally, the garment 310 may include any type(s) of wearable clothing or adornment suitable for wearing by a user and retaining one or more sensing modules as contemplated herein.

The garment 310 may include one or more designated areas 312 for positioning a module to sense a physiological parameter of the user 301 wearing the garment 310. One or more of the designated areas 312 may be specifically tailored for receiving a module 320 therein or thereon. For example, a designated area 312 may include a pocket structurally configured to receive a module 320 therein. Also or instead, a designated area 312 may include a first fastener configured to cooperate with a second fastener disposed on a module 320. One or more of the first fastener and the second fastener may include at least one of a hook-and-loop fastener, a button, a clamp, a clip, a snap, a projection, and a void.

The designated areas 312 may include at least one of a torso region, a spinal region, an extremity region (e.g., one or more of an arm region such as a sleeve, and a leg region such as a pant leg), a waistband region, a cuff region, and so on. Also or instead, one or more of the designated areas 312 may include at least a region adjacent to one or more muscle groups of the user 301—e.g., muscle groups including at least one of the pectoralis major, latissimus dorsi, biceps brachii, and so on. By way of example, the designated areas 312 may include at least one of the following: a wrist, an ankle, a bicep, a waist, a side torso (e.g., an area contained under a side strap of a bra or the like), a chest area (e.g., a location known to provide usable ECG data such as center chest), a finger, one or more locations on or around the head (e.g., forehead, neck, ear, and the like), and so on.

By placing a pocket or the like in one of these designated areas 312, a position of a module 320 can be controlled, and where an RFID tag, sensor, or the like is used, the designated area 312 can specifically sense when a module 320 is positioned there for monitoring, and can communicate the detected location to any suitable control circuitry. In this manner, a garment 310 may facilitate the installation of modules 320 in many different, discrete locations, the placement of which can be controlled by the configuration of the garment 310, and the use of which can be automatically detected when corresponding control modules 320 are placed there for use. Also or instead, the garment 310 may facilitate the placing of the modules 320 over relatively large regions of the garment 310. For example, a garment 310 may include a relatively large region (in terms of surface area) where a module 320 can be affixed or otherwise secured, e.g., by loops, straps, buttons, sheets of hook-and-loop fasteners, and so forth.

In general, each designated area 312 may include a pocket such as any of those described above, or any other mounting fixture or combination of fixtures. Where a pocket is used, the pocket may be configured as described above to preferentially urge a module 320 within the pocket toward the user's skin under normal pressure. Without limiting the generality of the foregoing, this may generally include an exterior layer of the pocket that is less elastic than an interior surface of the pocket so that when circumferential tension is applied (e.g., when the garment 310 is donned), the pocket preferentially urges a contact surface of the sensor inward toward the intended target surface with at least a predetermined normal force (when the garment 310 is properly sized for the user). In this respect, it will be understood that although some variation in normal force among users and garments is inevitable, typical tensions for comfortable use of properly fitted athletic wear are generally known, and adequate contact force to obtain a high quality physiological signal is generally known, and in any event readily observable in acquired data. As such, adequate circumferential tensions and resulting normal contact forces needed to promote good contact between sensing regions of the module 320 (such as LEDs, capacitive touch sensors, photodiodes, and the like) and the user's skin may readily be determined, and can advantageously facilitate the use of wrist-worn sensor housings such as those described above with one of the garments 310 described herein for off-wrist monitoring if/when desired.

In one aspect, the designated areas 312 may usefully be positioned where reinforcing elastic bands are typically provided on garments, e.g., around the mid-torso for a sports bra, around the waist on shorts or underwear, or on the sleeves of a t-shirt. In one aspect, the designated areas 312 may also usefully be positioned according to the intended physiological measurement, e.g., near major arteries suitable for heart rate detection using photoplethysmography. In one aspect, the garment 310 may usefully distribute these designated areas 312 (and supporting infrastructure such as wired connectors, location identification tags, and the like) at the intersection of regions where good physiological signals can be obtained and regions where adequate normal forces for good sensor contact can be generated by clothing. For example, this may include the ankles, the waist, the mid-torso, the biceps, the wrists, the forehead, and so on.

The garment 310 may also or instead incorporate other infrastructure 315 to cooperate with a module 320. For example, the garment infrastructure 315 may include infrastructure 315 related to ECG devices, such as ECG pads (or otherwise electrically-conductive sensor pads and/or electrodes that connect to the module 320, controller 330, and/or another component of the system 300), lead wires, and the like. By way of further example, the garment infrastructure 315 may include wires or the like embedded in the garment 310 to facilitate wired data or power transfer between installed modules 320 and other system components (including other modules 320). The infrastructure 315 may also or instead include integrated features for, e.g., powering modules, supporting data communications among modules, and otherwise supporting operation of the system 300. The infrastructure 315 may also or instead include location or identification tags or hardware, a power supply for powering modules 320 or other hardware, communications infrastructure as described herein, a wired intra-garment network, or supplemental components such as a processor, Global Positioning System (GPS) hardware/software, a timing device, e.g., for synchronizing signals from multiple garments, a beacon for synchronizing signals among multiple modules 320, and so forth. More generally, any hardware, software, or combination of these suitable for augmenting operation of the garment 310 and a physiological monitoring system using the garment 310 may be incorporated as infrastructure 315 into the garment 310 as contemplated herein.

The modules 320 may generally be sized and shaped for placement on or within the one or more designated areas 312 of the garment 310. For example, in certain implementations, one or more of the modules 320 may be permanently affixed on or within the garment 310. In such instances, the modules 320 may be washable. Also or instead, in certain implementations, one or more of the modules 320 may be removable and replaceable relative to the garment 310. In such instances, the modules 320 need not be washable, although a module 320 may be designed to be washable and/or otherwise durable enough to withstand a prolonged period of engagement with a designated area 312 of the garment 310. A module 320 may be capable of being positioned in more than one of the designated areas 312 of the garment 310. That is, one or more of the plurality of modules 320 may be configured to sense data using a physiological sensor 322 in a plurality of designated areas 312 of the garment 310.

Removable and replaceable modules 320 may provide several advantages such as ease of garment care (e.g., washing) and power management (e.g., removal for recharging). Furthermore, removability may facilitate replacement and/or repositioning of modules within the garment 310 for different sensing activities or other reconfigurations, replacement of damaged or defective modules 320, and so forth.

A module 320 may include one or more physiological sensors 322 and a communications interface 324 programmed to transmit data from at least one of the physiological sensors 322. For example, the physiological sensors 322 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, or other sensor or combination of sensors and the like useful for physiological monitoring, environmental monitoring, or other monitoring as described herein. In one aspect, the physiological sensors 322 may include a conductivity sensor or the like used for electromyography, electrocardiogramactroencephalography, or other physiological sensing based on electrical signals. The data received from the physiological sensors 322 may include at least one of heart rate data and/or similar data related to blood flow (e.g., from PPG sensors), muscle oxygen saturation data, temperature data, movement data, position/location data, environmental data, temporal data, blood pressure data, and so on.

In one aspect, a module 320 may be configured for use on multiple body locations. For example, the module 320 may be one of the wrist-worn sensors described above. The module 320 may be adapted for use with a garment 310 in various ways. In one aspect, the module 320 may have relatively smooth, continuous exterior surfaces to facilitate sliding into and out of a pocket, such as any of the pockets described herein, or any other suitable retaining structure(s). In another aspect, an LED and/or sensor region may protrude from a surface of the module 320 sufficiently to extend beyond a restraining garment material and into a contact surface of a user. The module 320 may also include hardware to facilitate such uses. For example, a module 320 may usefully incorporate a contact sensor for detecting contact with a user. However, the exposed contact surfaces of the module 320 may be different when retained by a wrist strap (or other limb strap) than when retained by a garment pocket. To facilitate multiple retaining modes, the module 320 may usefully incorporate two or more contact sensors (such as capacitive sensors or other touch sensors, switches, or the like) at two different locations, each positioned to detect contact with a wearer in a different retaining mode. For example, a module 320 may include a capacitive sensor adjacent to an optical sensing system that contacts the user's skin when the module 320 is retained with a wrist strap. The module 320 may also or instead optically detect contact when the capacitive sensor is covered by a garment fabric or the like that prevents direct skin contact, or a second capacitive sensor may be placed within another region exposed by the garment 310 retaining system. In another aspect, the garment 310 may include a capacitive sensor that provides a signal to the module 320, or to some other system controller or the like, when a region of the garment near the module 320 is in contact with a user's skin.

In one aspect, the physiological sensors 322 may include a heart rate monitor or pulse sensor, e.g., where heart rate is optically detected from an artery, such as the radial artery. In one embodiment, the garment 310 may be configured such that a module 320 is positioned on a user's wrist, where a physiological sensor 322 of the module 320 is secured over the user's radial artery or other blood vessel. Secure connection and placement of a pulse sensor over the radial artery or other blood vessel facilitates measurement of heart rate, pulse oxygen, and the like. It will be understood that this configuration is provided by way of example only, and that other sensors, sensor positions, and monitoring techniques may also or instead be employed without departing from the scope of this disclosure.

In some embodiments, heart rate data may be acquired using an optical sensor coupled with one or more light emitting diodes (LEDs), all in contact with the user 301. To facilitate optical sensing, the garment 310 may be designed to maintain a physiological sensor 322 in secure, continual contact with the skin, and reduce interference of outside light with optical sensing by the physiological sensor 322.

Thus, certain embodiments include one or more physiological sensors 322 configured to provide continuous measurements of heart rate using photoplethysmography or the like. The physiological sensor 322 may include one or more light emitters for emitting light at one or more desired frequencies toward the user's skin, and one or more light detectors for received light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 322 may also or instead provide at least one of continuous motion detection, environmental temperature sensing, electrodermal activity (EDA) sensing, galvanic skin response (GSR) sensing, and the like.

The system 300 may include different types of modules 320. For example, a number of different modules 320 may each provide a particular function. Thus, the garment 310 may house one or more of a temperature module, a heart rate/PPG module, a muscle oxygen saturation module, a haptic module, a wireless communication module, or combinations thereof, any of which may be integrated into a single module 320 or deployed in separate modules 320 that can communicate with one another. Some measurements such as temperature, motion, optical heart rate detection, and the like, may have preferred or fixed locations, and pockets or fixtures within the garment 310 may be adapted to receive specific types of modules 320 at specific locations within the garment 310. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 300 may include two or more modules 320 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 324 may be any as described herein, for example including any of the features of the network interface 304 described above. The communications interface 324 may be a separate device that provides the ability for the modules 320 to communicate with one another and/or with other components of the system 300), or there may be a central module that communicates with other modules 320 (or with another component of the system 300). It will be understood that communications may usefully be secured using any suitable encryption technology in order to ensure privacy and security of user data. This may, for example, include encryption for local (wired or wireless) communications among the modules 320 and/or controller 330 within the garment 310. This may also or instead include encryption for remote communications to a server and other remote resources. In one aspect, the garment 310 and/or controller 330 may provide a cryptographic infrastructure for securing local communications, e.g., by managing public/private key pairs for use in asymmetric encryption, authentication, digital signatures, and so forth. The keys for this infrastructure may also or instead be managed by an external, trusted third party.

The controller 330 may be configured, e.g., by computer executable code or the like, to determine a location of the module 320. This may be based on contextual measurements such as accelerometer data from the module 320, which may be analyzed by a machine learning model or the like to infer a body position. In another aspect, this may be based on other signals from the module 320. For example, signals from sensors such as photodiodes, temperature sensors, resistors, capacitors, and the like may be used alone or in combination to infer a body position. In another aspect, the location may be determined based on a proximity of a module 320 to a proximity sensor, RFID tag, or the like at or near one of the designated areas 312 of the garment 310. Based on the location, the controller 330 may adapt operation of the module 320 for location-specific operation. This may include selecting filters, processing models, physiological signal detections, and the like. It will be understood that operations of the controller 330, which may be any controller, microcontroller, microprocessor, or other processing circuitry, or the like, may be performed in cooperation with another component of the system 300 such as the processor 340 described herein, one or more of the modules 320, or another computing device. It will also be understood that the controller 330 may be located on a local component of the system 300 (e.g., on the garment 310, in a module 320, and so on) or as part of a remote processing facility 350, or some combination of these. Thus, in an aspect, a controller 330 is included in at least one of the plurality of modules 320. And, in another aspect, the controller 330 is a separate component of the garment 310, and serves to integrate functions of the various modules 320 connected thereto. The controller 330 may also or instead be remote relative to each of the plurality of modules 320, or some combination of these.

Location detection (i.e., of the modules 320 and/or physiological sensors 322) may also usefully be recorded and used in a number of ways by a human user and/or by the system 300. For example, a detected location may be stored, along with the corresponding garment, so that a user can retrieve a placement history and replace the module 320 to a previous location for a particular garment as desired. In another aspect, the detected location may be used by the system 300 to analyze data and make garment specific recommendations. For example, the system 300 may evaluate the quality of a signal, e.g., using any conventional metrics such as signal-to-noise ratio, or using quality metrics more specific to physiological signals such as correlation to an expected signal or pulse shape, consistency with a rate or magnitude typical for a sensor, pulse-to-pulse consistency for a particular user, or any other measure of signal quality using statics, machine learning, digital signal processing techniques, or the like. A quality metric, however derived, may be used in turn to recommend specific placements of a module 320 on a garment 310 for a user, or to recommend a particular garment 310 for the user. Thus, for example, after acquiring data over a range of garments and activities, the system 300 may generate a user-actionable recommendation such as, “It appears that when you are jogging, the most accurate heart rate signals can be obtained when you are wearing an XL shirt model number xxxxxx. You may wish to wear this shirt for active workouts, and you may wish to purchase more of this type of shirt for regular use.” As another example, the user-actionable recommendation may suggest: “It appears that one of your modules is not obtaining accurate temperature readings when located on your sleeve elastic band. You may wish to try a different location for this module, or to try a different garment.” More generally, data quality may be measured for a number of different modules at different locations in different garments during different activities, and this data may be used to generate customized recommendations for a user on a per-garment and per-location basis. These recommendations may also be tailored to specific activity types where this data is accurately recorded by the system 300, either from user input, automatic detection, or some combination of these.

The controller 330 may be configured to control one or more of (i) sensing performed by a physiological sensor 322 of the module 320 and (ii) processing by the module 320 of the data received from a physiological sensor 322. That is, in certain aspects, the combination of sensors in the module 320 may vary based on where it is intended to be located on a garment 310. In another aspect, processing of data from a module 320 may vary based on where it is located on a garment 310. In this latter aspect, a processing resource such as the controller 330 or some other local or remote processing resource coupled to the module 320 may detect the location and adapt processing of data from the module 320 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, a GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 310, and then use this detected location to select a suitable model for activity recognition. This technique may similarly be applied to calibration models, physiological signals processing models, and the like, or to otherwise adapt processing of signals from a module 320 based on the location of the module 320.

Determining the location of a module 320 may include receiving a sensed location for the module 320. The sensed location may be provided by a proximity detection circuit such as a near-field-communication (NFC) tag, an (active or passive) RFID tag, a capacitance sensor, a magnetic sensor, an electrical contact, a mechanical contact, and the like. Any corresponding hardware for such proximity detections may be disposed on the module 320 and the garment 310 for communication therebetween to detect location when appropriate. For example, in one aspect, an NFC tag may be disposed on or within the garment 310, and the module may include sensor 322 such as an NFC tag sensor that can detect the tag and read any location-specific information therefrom. Proximity detection may also or instead be performed using capacitively detected contact, electromagnetically detected proximity, mechanical contact, electrical coupling, and the like. In this manner, a garment 310 may provide information to an installed module 320 to inform the module 320, among other things, where the module 320 is located, or vice-versa.

Thus, communication between a module 320 and the garment 310 (or a processor of the garment 310) may be used to determine the location of a module 320 on the garment 310. Communication of location information may be enabled using active techniques, passive techniques, or a combination thereof. For example, a thin, flexible, cheap, washable NFC tag may be sewn into the garment 310 in various locations where a module 320 may be placed. When a module 320 is placed in the garment 310, the module 320 may query an adjacent NFC tag to determine its location. Furthermore, the NFC technique or other similar techniques may provide other information to the module 320, including details about the garment 310 such as the size, whether it is a gender specific piece, the manufacturer information, model or serial number of the garment, stock keeping unit (SKU), and more. Similarly, the tag may encode a unique identifier for the garment 310 that can be used to obtain other relevant information using an online resource. The module 320 may also or instead advertise information about itself to the garment 310 so that the garment 310 can synchronize processing with other modules 320, synchronize communication among modules 320, control or condition signals from the module 320, and so forth. The module 320 can then configure itself within the context of the current garment 310 and associated modules 320, and/or to perform certain types of monitoring or data processing.

Determining the location of a module 320 may also or instead be based, at least in part, on an interpretation of the data received from a physiological sensor 322 of the module 320. By way of example, movement of a module 320 as detected by a sensor may provide information that can be used to predict a position on or within the garment 310. Also or instead, the type of data that is being received from a module 320 may indicate where the module 320 is located on the garment 310. For example, locations may produce unique signatures of acceleration, gyroscope activity, capacitive data, optical data, temperature data, and the like, depending on where the module 320 is located, and this data may be fused and analyzed in any suitable manner to obtain a location prediction.

According to the foregoing, determining the location of a module 320 may also or instead include receiving explicit input from the user 301, which may identify one of the designated areas on the garment 310, or a general area of the body (e.g., left wrist, right ankle, and so forth). Because the location of the module 320 relative to the garment 310 may be determined from an analysis of a plurality of data sources, the system 300 may include a component (e.g., the processor 340) that is configured to reconcile one or more potential sources of location of information based on expected reliability, measured quality of data, express user input, and so forth. A prediction confidence may also usefully be generated in this context, which may be used, for example, to determine whether a user should be queried for more specific location information. More generally, any of the foregoing techniques may be used along or in combination, along with a failsafe measure the requests user input when location cannot confidently be predicted. Also or instead, a user may explicitly specify a prediction preemptively, or as an override to an automatically generated prediction.

Once determined using any of the techniques above, the location of a module 320 may be transmitted for storage and analysis to a remote processing facility 350, a database 360, or the like. That is, in addition to the module 320 using this information locally to configure itself for the location in which it is worn, the module 320 may communicate this information to other modules 320, peripherals, or the cloud. Processing this information in the cloud may help an organization determine if a module 320 has ever been installed on a garment 310, which locations are most used, and how modules 320 perform differently in different locations. These analytics may be useful for many purposes, and may, for example, be used to improve the design or use of modules 320 and garments 310, either for a population, for a user type, or for a particular user.

As stated above, the system 300 may further include a processor 340 and a memory 342. In general, the memory 342 may bear computer executable code configured to be executed by the processor 340 to perform processing of the data received from one or more modules 320. One or more of the processor 340 and the memory 342 may be located on a local component of the system 300 (e.g., the garment 310, a module 320, the controller 330, and the like) or as part of a remote processing facility 350 or the like as shown in the figure. Thus, in an aspect, one or more of the processor 340 and the memory 342 is included on at least one of the plurality of modules 320. In this manner, processing may be performed on a central module, or on each module 320 independently. In another aspect, one or more of the processor 340 and the memory 342 is remote relative to each of the plurality of modules 320. For example, processing may be performed on a connected peripheral device such as smartphone, laptop, local computer, or cloud resource.

The memory 342 may store one or more algorithms, models, and supporting data (e.g., parameters, calibration results, user selections, and so forth) and the like for transforming data received from a physiological sensor 322 of the module 320. In this manner, suitable models, algorithms, tuning parameters, and the like may be selected for use in transforming the data based on the location of the module 320 as determined by the controller 330 and/or processor 340 as described herein. By way of example, algorithms that convert data from an accelerometer in a module 320 into activity data or a count of a user's steps may be different depending on whether the module 320 is worn on the user's wrist or on the user's waist band. Similarly, the intensity of an LED and corresponding sensitivity of a photodetector may be different for a PPG device placed on the wrist or the thigh. Thus, the module 320 may self-configure for a location by controlling one or more of sensor types, sensor parameters, processing models, and so forth based on a detected location for the module 320.

Selection of an algorithm may also or instead include an analysis of one or more of the sensor data, metadata, and the like. By way of example, an algorithm may be selected at least in part based on metadata received from one of the module 320 and the garment 310. This metadata may be derived from communication between the module 320 and the garment 310—e.g., between a tag and tag reader for exchanging information therebetween. For example, the garment 310 may include, e.g., stored in a tag such as an NFC tag or other wirelessly readable data source, garment-specific metadata that is readable by or otherwise transmittable to one or more of the plurality of modules 320, the controller 330, and the processor 340. Such garment-specific metadata may include at least one of a type of garment 310, a size of the garment 310, garment dimensions, a gender configuration of the garment 310, a manufacturer, a model number, a serial number, a SKU, a material, fit information, and so on. In one aspect, this information may be provided with one or more of the location identification tags described herein. In another aspect, the garment 310 may include an additional tag at a suitable location (e.g., near or accessible to a processor or controller) that provides garment-specific information while other tags provide location-specific information.

The metadata may also or instead include at least one of a gender of the user 301, a weight of the user 301, a height of the user 301, an age of the user 301, metadata associated with the garment 310 (e.g., the garment size, type, material, etc.), and the like. The metadata may be derived, at least in part, from user-provided input, or otherwise from information derived from the user 301 such as a user's account information as a participant in the system 300. By way of example, a processing algorithm may be selected depending on the material of the garment 310 as communicated by its serial or model number in an identification tag, the physiology of the user 301 as implied by the garment size, and so on. The metadata may also or instead be used to verify the authenticity of the garment 310, and otherwise control access to the garment 310 and/or modules 320 coupled to the garment 310. In one aspect, metadata (e.g., size, material) may be encoded directly into the garment metadata. In another aspect, the garment 310 may publish a unique identifier that can be used to retrieve related information from a manufacturer or other data source. This latter approach advantageously permits correlation of garment-specific data with other user-specific data such as height, weight, body composition, and so forth.

Simply knowing a priori where a module 320 is positioned may allow for the use of algorithms that have been developed to perform optimally in that particular location. This can relieve a significant computational burden otherwise borne by the module 320 to analytically evaluate location based on available signals. Other information may also or instead be used to select an optimal algorithm. For example, based on the gender or dimensions of a garment, the algorithm may employ different models or different model parameters.

The processor 340 may be configured to assess the quality of the data received from a physiological sensor 322 of the module 320. For example, the processor 340 may be configured to provide, based on the quality of the data, a recommendation regarding at least one of the location of a module 320 and an aspect of the garment 310 (e.g., size, fit, material, and so on). For example, the processor 340 may be configured to detect when the garment does not properly fit the wearer for acquisition of physiological data, for example, by detecting when a module is moving (e.g., from accelerometer data) but data quality is poor or absent for a sensed physiological signal. In general, the garment 310 may store its own identifier and/or metadata, e.g., as described herein, or garment identification data may be stored in tags, e.g., at designated areas 312 of the garment 310. The processor 340 may be configured to use this garment identification information and/or metadata to provide a recommendation regarding a different garment 310 for the user 301, or for an adjustment to the current garment 310. For example, if a particular garment 310 seems to result in low-quality data, the user 301 could be encouraged to select an alternative size, or to make some other adjustment. Moreover, data on how many times a garment 310 is used may be gathered and used to inform business decisions, for example, which garments 310 provide the highest-quality data, and which garments 310 are most preferred by users 301.

The system 300 may further include a database 360, which may be located remotely and in communication with the system 300 via the data network 302. The database 360 may store data related to the system 300 such as any discussed herein—e.g., sensed data, processed data, transformed data, metadata, physiological signal processing models and algorithms, personal activity history, and the like. The system 300 may further include one or more servers 370 that host data, provide a user interface, process data, and so forth in order to facilitate use of the modules 320 and garments 310 as described herein.

It will be appreciated that the garment 310, modules 320, and accompanying garment infrastructure and remote networking/processing resources, may advantageously be used in combination to improve physiological monitoring and achieve modes of monitoring not previously available.

One or more of the devices and systems described herein may include circuitry for both wireless charging and wireless data transmission, e.g., where the corresponding circuits can operate independently from one another, and where the corresponding antennae are located proximal to one another (for instance, the circuitry for wireless charging and the circuitry for wireless data transmission may include separate coils disposed substantially along the same plane, or otherwise in relative close proximity in a device or system). In such aspects, one or more measures may be taken so that a wireless data transfer process does not interfere with a wireless power transfer process, more specifically by coupling the data circuitry into the electromagnetic field for the wireless power transfer in a manner that alters the resonant frequency or otherwise destructively interferes with power transfer, thereby decreasing efficiency when charging a device. For example, a switch may be included to disable circuitry for data transmission when certain wireless charging activity is present, thereby allowing for relatively unimpeded and efficient wireless charging of a device. The switch may also be operable to enable operation of data transmission circuitry when certain wireless charging activity is not present.

Thus, for example, in the context of a physiological monitor, such as any of those described herein, the physiological monitor may include both a wireless power receiver (or similar) and a wireless data tag reader (or similar). In general, these sub-systems may conform to one or more Near Field Communication (NFC) specifications for protocols and physical architectures, or any other standards suitable for wireless power and data transmission. The power circuitry may be used, e.g., to charge a battery on the physiological monitor so that the device can be recharged without physically connecting to a power source. The data circuitry may be used, e.g., as a wireless data tag reader or the like to read data from nearby data sources such as identification tags in user apparel and the like. In general, the physiological monitor may include separate circuitry (separate coils) for these wireless power and data systems, such as separate processing circuitry and/or separate antennae. The antennae may be disposed substantially along the same plane of the physiological monitor (e.g., with one coil disposed substantially inside or adjacent to the other). In one aspect, the antennae may be in parallel planes, however, it will be noted that distance tolerances for NFC standard devices are relatively small, and the physically housing for these antennae will preferably enforce an identical or substantially identical distance for both antennae in such architectures. In this context, the positions of the antennae may be as close to parallel as possible within reasonable manufacturing tolerances, or as close to parallel as possible when disposed on two different layers of a shared printed circuit board, or preferably, when disposed on a single layer of a shared printed circuit board. The physiological monitor may further include a switch (e.g., a radio frequency (RF) switch or the like) in-line with the coil for the wireless data tag reader to disable the wireless data tag reader when power is being received to mitigate any effects on the efficiency of the wireless power transfer process. In particular, the switch may be configured to open when power is being received, and may be configured to close when the physiological monitor is looking for data tag to read.

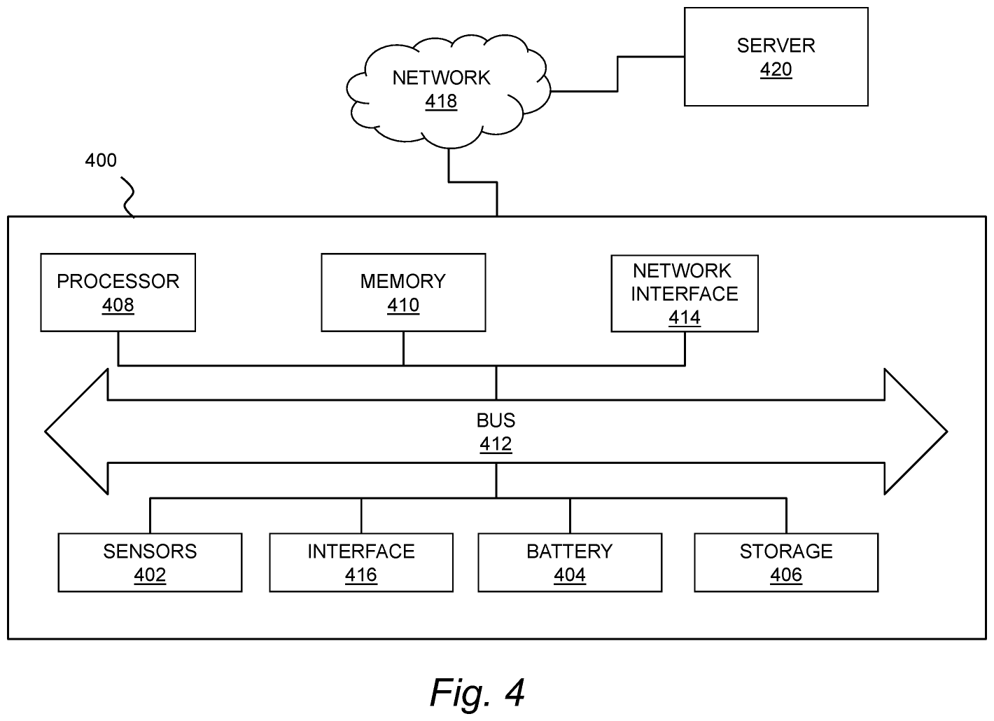

FIG. 4 is a block diagram of a computing device 400. The computing device 400 may, for example, be a device used for continuous physiological monitoring, or any other device supporting a physiological monitor in the systems and methods described herein. The device may also or instead be any of the local computing devices described herein, such as a desktop computer, laptop computer, smartphone. The device may also or instead be any of the remote computing resources described herein, such as a web server, a cloud database, a file server, an application server, or any other remote resource or the like. While described as a physical device, it will be understood that the exemplary computing device 400 may also or instead be realized as a virtual computing device such as a virtual machine executing a web server or other remote resource in a cloud computing platform. In general, the device 400 may include one or more sensors 402, a battery 404, storage 406, a processor 408, memory 410, a network interface 414, and a user interface 416, or virtual instances of one or more of the foregoing.

The sensors 402 may include any sensor or combination of sensors suitable for heart rate monitoring as contemplated herein, as well as sensors 402 for detecting calorie burn, position (e.g., through a Global Positioning System or the like), motion, activity and so forth. In one aspect, this may include optical sensing systems including LEDs or other light sources, along with photodiodes or other light sensors, that can be used in combination for photoplethysmography measurements of heart rate, pulse oximetry measurements, and other physiological monitoring.

The sensors 402 may also or instead include one or more sensors for activity measurement. In some embodiments, the system may include one or more multi-axes accelerometers and/or gyroscope to provide a measurement of activity. In some embodiments, the accelerometer may further be used to filter a signal from the optical sensor for measuring heart rate and to provide a more accurate measurement of the heart rate. In some embodiments, the wearable system may include a multi-axis accelerometer to measure motion and calculate distance. Motion sensors may be used, for example, to classify or categorize activity, such as walking, running, performing another sport, standing, sitting or lying down. The sensors 402 may, for example, include a thermometer for monitoring the user's body or skin temperature. In one embodiment, the sensors 402 may be used to recognize sleep based on a temperature drop, Galvanic Skin Response data, lack of movement or activity according to data collected by the accelerometer, reduced heart rate as measured by the heart rate monitor, and so forth. The body temperature, in conjunction with heart rate monitoring and motion, may be used, e.g., to interpret whether a user is sleeping or just resting, as well as how well an individual is sleeping. The body temperature, motion, and other sensed data may also be used to determine whether the user is exercising, and to categorize and/or analyze activities as described in greater detail below. In another aspect, the sensors 402 may include one or more contact sensors, such as a capacitive touch sensor or resistive touch sensor, for detecting placement of a physiological monitor for use on a user. More generally, the sensors 402 may include any sensor or combination of sensors suitable for monitoring geographic location, physiological state, exertion, movement, and so forth in any manner useful for physiological monitoring as contemplated herein.

The battery 404 may include one or more batteries configured to allow continuous wear and usage of the wearable system. In one embodiment, the wearable system may include two or more batteries, such as a removable battery that may be removed and recharged using a charger, along with an integral battery that maintains operation of the device 400 while the main battery charges. In another aspect, the battery 404 may include a wireless rechargeable battery that can be recharged using a short range or long range wireless recharging system.

The processor 408 may include any microprocessor, microcontroller, signal processor or other processor or combination of processors and other processing circuitry suitable for performing the processing steps described herein. In general, the processor 408 may be configured by computer executable code stored in the memory 410 to provide activity recognition and other physiological monitoring functions described herein.

In general the memory 410 may include one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, optical disks, USB flash drives), and the like. In one aspect, the memory 410 may include a computer system memory or random access memory, such as DRAM, SRAM, EDO RAM, and the like. The memory 410 may include other types of memory as well, or combinations thereof, as well as virtual instances of memory, e.g., where the device is a virtual device. In general, the memory 410 may store computer readable and computer-executable instructions or software for implementing methods and systems described herein. The memory 410 may also or instead store physiological data, user data, or other data useful for operation of a physiological monitor or other device described herein, such as data collected by sensors 402 during operation of the device 400.

The network interface 414 may be configured to wirelessly communicate data to a server 420, e.g., through an external network 418 such as any public network, private network, or other data network described herein, or any combination of the foregoing including, e.g., local area networks, the Internet, cellular data networks, and so forth. Where the device is a physiological monitoring device, the network interface 414 may be used, e.g., to transmit raw or processed sensor data stored on the device 400 to the server 420, as well as to receive updates, receive configuration information, and otherwise communicate with remote resources and the user to support operation of the device. More generally, the network interface 414 may include any interface configured to connect with one or more networks, for example, a Local Area Network (LAN), a Wide Area Network (WAN), the Internet, or a cellular data network through a variety of connections including, but not limited to, standard telephone lines, LAN or WAN links (for example, 802.11, T1, T3, 56 kb, X.25), broadband connections (for example, ISDN, Frame Relay, ATM), wireless connections, or some combination of any or all of the above. The network interface 412 may include a built-in network adapter, network interface card, PCMCIA network card, card bus network adapter, wireless network adapter, USB network adapter, modem or any other device suitable for interfacing the computing device 400 to any type of network capable of communication and performing the operations described herein.

The user interface 416 may include any components suitable for supporting interaction with a user. This may, for example, include a keypad, display, buzzer, speaker, light emitting diodes, capacitive touch sensors or pads, and any other components for receiving input from, or providing output to, a user. In one aspect, the device 400 may be configured to receive tactile input, such as by responding to sequences of taps on a surface of the device to change operating states, display information and so forth. The user interface 416 may also or instead include a graphical user interface rendered on a display for graphical user interaction with programs executing on the processor 408 and other content rendered by a physical display of device 400.

### System for Dynamic Stress Scoring

Techniques are described herein for managing stress, including techniques for quantifying stress to obtain a continuous or periodic quantitative, objective measure of stress, e.g., using a physiological monitoring device and/or any other suitable contextual data, as well as techniques to analyze such quantitative stress data to providing coaching or other guidance, feedback, recommendations, and the like. In general, stress, as described herein, may include any measurable physiological and/or psychological response to various stressors or stimuli such as physical stressors, environmental stressors, and psychological stressors.

In one aspect, stress may be physiological stress in response to physiological stressors that physically induce stress for a user. For example, physiological stressors may include muscular and/or cardiovascular activity such as workouts, sports activity, or other routine or one-off activities that tax an individual due to physical load. Physical stressors may also or instead include illness, exposure (e.g., to toxins, allergens, drugs, alcohol, and the like), dehydration, inadequate or low quality sleep, and the like that tax an individual due to behavior or circumstance. In another aspect, stress may be environmental stress in response to environmental stressors such as sound, odors, lighting, temperature, humidity, and so forth, any of which may tax an individual due to exposure. In another aspect, stress may be emotional stress in response to psychological stressors such as work pressure, financial difficulties, relationship problems, and/or health concerns that tax an individual due to psychological or emotional conditions.

In some instances, a particular stimulus may create one or more types of stress. For example, heat or cold may be an environmental stressor where contact with cold (e.g., rapid immersion in cold water) or heat (e.g., touching a hot metallic surface) induces a short term physiological response such as a fight-or-flight response. Heat or cold may also, however, be a physiological stressor, e.g., where a user has a physiological response to prolonged heat or cold exposure. Similarly, poor sleep is known to result in suppressed immune function and emotional dysregulation. These may be physiological stressors, but they may also result in greater emotional stress over the course of a day, thus creating an additional psychological stressor for a user. A user may be subject to other compound or complex stressors. For example, excessive alcohol consumption may impose physiological stress as the body seeks to metabolize and dispose of consumed alcohol. This may also impair sleep quality, which may lead to additional psychological stresses on subsequent days. More generally, a user may be subject to numerous concurrent or sequential stressors of different forms that may contribute to cumulative stress over the course of hours or days.

Where possible, it may be useful to isolate and monitor various contributors to an objectively measured user stress in order to provide more suitable coaching feedback. For example, when faced with a perceived threat, the brain sends signals to the hypothalamus, which activates the sympathetic nervous system. This triggers the release of hormones like adrenaline and cortisol, which increase heart rate, breathing rate, and blood pressure. In small amounts, this type of stress can helpfully motivate action to address a challenging situation. However, chronic or excessive stress of this type can lead to negative effects such as anxiety, depression, fatigue, or a weakened immune system. At the same time, physiological stress responses may be a healthy reaction to vigorous activity. Incremental monitoring of various stress responses can support intra-day updates to performance metrics, coaching recommendations, and the like, e.g., to support updated strain calculations, and/or real time recommendations concerning new or ongoing exercise, diet, and so forth.

To this end, the characterization of stress may be aided by various forms of contextual data for a user. For example, contextual information may include environmental conditions such as temperature, air quality, and the like, which may be measured (e.g., by measuring ambient temperature with a wearable device) or obtained from third party sources (e.g., based on reported air quality metrics) and used to estimate environmental contributors to measured stress. In another aspect, contextual information may include motion data, e.g., where user activity is monitored using motion sensors on a wearable device, and used to isolate aspects of stress that are due to physical activity. For example, where a user has an elevated heart rate, this may be attributed to exercise where concurrent motion is detected by motion sensors of a wearable device, particularly where the motion can be identified as relating to a particular type of activity (e.g., swimming, biking, jogging, weight lifting, playing tennis, etc.).

In another aspect, contextual data may include self-reported user activity (e.g., diet, alcohol consumption, smoking, etc.), which may be used to estimate related physiological responses that might manifest in stress data. In another aspect, contextual data may include activity measured by, or inferred by, the physiological monitor. For example, where a wearable monitor can measure core temperature or blood pressure, these physiological metrics may be used to assess possible health issues that might impact measured stress. As another example, where physiological data can be used to identify sleep patterns or activity, this may be used to inform stress calculations. For example, an elevated physiological response (e.g., increased heart rate) during sleep, and in particular, within intervals confined to dreaming phases of sleep, may be interpreted as psychological stress, while prolonged changes in heart rate or heart rate variability over the course of longer sleep interval may be an indication of physical stress in the form of recovery, illness, or the like.

To the extent that context permits, a calculated stress score may include adjustments for context, or multiple scores for different forms of stress, or may include an indicator of various contributors to an aggregated or instantaneous stress score that is measured for a user. As a significant advantage, the techniques described herein can facilitate monitoring and management of psychological and physiological stressors in real time or near real time. As another advantage, the techniques described herein can facilitate the separate measurement of different contributors to a calculated stress responses, e.g., to permit the isolation and analysis of contributions by physical, environmental, and psychological stimuli.

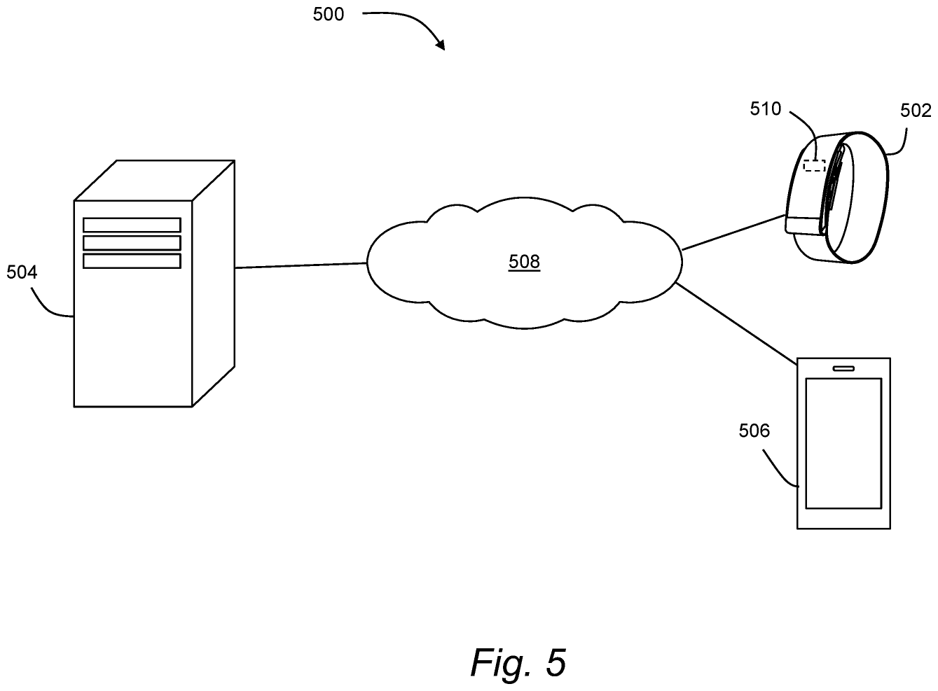

FIG. 5 shows a system for dynamic stress monitoring. In general, a physiological monitoring system 500 may include a wearable device 502 such as any of the physiological monitors described herein, a remote resource 504 such as any of the servers or other remote processing resources described herein, and a user device 506 such as any of the user devices described herein, along with a data network 508 interconnecting these devices in a communicating relationship.

The wearable device 502 may continuously monitor, measure, and/or calculate physiological parameters such as heart rate, heart rate variability, temperature, electrical properties (e.g., electrodermal activity and the like), blood pressure, stress, and motion, and transmit this data to a remote resource 504 over the data network 508. Based on this data, the remote resource may calculate metrics such as a daily sleep score (evaluating a prior night's sleep), a daily strain score (evaluating a prior day's strain), and/or a daily recovery score (evaluating readiness for strain), for example using techniques described in U.S. Pat. No. 10,264,982 issued on Apr. 23, 2019, the entire content of which is hereby incorporated by reference. This server-based approach advantageously permits offloading of data-intensive and computationally intensive processing to a remote server or other suitably capable computing system, e.g., for metrics that are based on heart rate and motion data for an entire twenty four hour interval. However, this approach can be less effective for incremental updates to strain over the course of the day.

Thus, in one aspect the techniques described herein may advantageously be deployed to dynamically monitor stress over the course of the day, and update a user's metrics and coaching recommendations in real time, or as otherwise appropriate. More specifically, a dynamic stress monitor 510 may be deployed locally on the wearable device 502, or at some other convenient location (such as the user device 506) where it can perform frequent local calculations to dynamically report and/or update user information. This approach also advantageously facilitates quick detection of significant stress events so that suitable interventions can be recommended.

It will be understood that in this context, “dynamic” scoring refers to scoring that is performed incrementally between static, remote calculations performed, e.g., on a server or other remote resource for long intervals such as several hours or days. While dynamic scoring will generally entail a discrete calculation performed for a specific time period or interval, the scoring may be updated in any periodic or substantially continuous manner such that user can receive timely quantitative evaluations. For example, this may include updates that are as close to instantaneous as possible, so that they are experienced by the user in real time with little or no observable latency, or over a short time interval, e.g., once per second or once every few seconds, so that the user can compare the current dynamic score to the user's current subjective state. In another aspect, where the stress calculations are more computationally complex and/or are processed remotely, a current stress score may be calculated and updated for the user at an interval such as once per minute, or at some shorter or longer interval suitable for consumption by the user. This may also include changing the frequency, e.g., to update more frequently and/or provide more current calculations while a user is viewing the stress score. For example, the dynamic score may be updated once per minute, and also immediately (e.g., on demand) whenever a user checks the stress score. As another example, implementations may include providing a user with information related to an accumulation of stress over the course of the day or another time period, such as by presenting to the user their time spent (e.g., in minutes) in different stress zones throughout a day or another relevant time period. More generally, any quantity or frequency of updates that facilitates dynamic tracking of stress, and/or that supports feedback to the user on a current stress state, may be used to support tracking and reporting of a user's current state of subjective or physiological stress.

In another aspect, a system described herein may include a wearable physiological monitor including one or more sensors and a first processor configured to continuously acquire heart rate data for a user based on a signal from the one or more sensors; and one or more processors coupled in a communicating relationship with the wearable physiological monitor. The one or more processors may be configured by computer executable code to receive data from the wearable physiological monitor and to: calculate a heart rate variability metric for the user based on the heart rate data; calculate a heart rate metric for the user based on the heart rate data; determine a resting state of the user based on the heart rate data; and calculate a stress score for the user. The stress score may be calculated based on a weighted combination of the heart rate variability metric and the heart rate metric, where: the weighted combination uses a first weight for the heart rate metric based on the resting state of the user, and the weighted combination uses a second weight for the heart rate variability based on the resting state of the user. The system may further include a display device in communication with the one or more processors, the display device including a user interface configured to present a value to the user indicative of the stress score. In some aspects, at least one processor of the one or more processors is disposed on the display device. In some aspects, at least one processor of the one or more processors is disposed on a remote server. In some aspects, at least one processor of the one or more processors is disposed on a user device. In some aspects, a processor of the one or more processors is disposed on a remote server, and another processor of the one or more processors is disposed on a user device. In this manner, it will be understood that at least part of the stress score may be calculated using processing capabilities of both a user device and a remote server.

In another aspect, a system described herein includes: a wearable physiological monitor including one or more sensors and a first processor configured to continuously acquire data for a user based on a signal from the one or more sensors, the data including a heart rate, a heart rate variability, and a motion; and a second processor coupled in a communicating relationship with the wearable physiological monitor. The second processor may be configured by computer executable code to receive data from the physiological monitor and to: measure an aggregate heart rate of the user over an interval; determine whether the aggregate heart rate over the interval is within a predetermined range of a resting heart rate for the user; in response to determining that the aggregate heart rate is outside the predetermined range, calculate a stress score for the user over the interval based on the data from the physiological monitor acquired during the interval; and, in response to determining that the aggregate heart rate is within the predetermined range, calculate the stress score for the user based on the data from the physiological monitor acquired during the interval using a lower weighted contribution of the heart rate variability relative to the heart rate than a weighted contribution used when it is determined that the aggregate heart rate is outside the predetermined range. The system may further include a display device in communication with the second processor, the display device including a user interface configured to present a value to the user indicative of the stress score for the interval. The second processor may be disposed on one or more of: the wearable physiological monitor, the display device, and a remote server.

It will be understood that, when discussing heart rate and/or similar metrics herein (e.g., resting heart rate, heart rate variability, and the like), the term “aggregate” (such as the “aggregate heart rate” discussed in the preceding paragraph) shall include metrics that account for a plurality of measurements (e.g., over one or more specific time intervals). Thus, instead of analyzing individual data points, such an aggregate metric can be used to examine the overall characteristics or trends of a dataset. This aggregation process may involve combining, averaging, summing, or otherwise summarizing values within a dataset to derive a single “aggregate” metric. Such an aggregate metric may include without limitation at least one of an average, a median, a mode, a total, a range, a rate, a percentage, a proportion, and so on. An aggregate metric may also or instead include or otherwise account for one or more of a variance, a standard deviation, a coefficient of variation, and so forth.

In another aspect, a system disclosed herein includes a wearable physiological monitor including one or more sensors and a first processor configured to continuously acquire data for a user based on a signal from the one or more sensors, the data including a heart rate, a heart rate variability, and a motion; and a second processor coupled in a communicating relationship with the wearable physiological monitor. The second processor may be configured by computer executable code to receive data from the physiological monitor and to: determine a sleep state of the user; in response to determining that the sleep state is an awake state, calculate a stress score for the user over an interval based on the data received from the physiological monitor; and, in response to determining that the sleep state is an asleep state, calculate the stress score for the user based on the data received from the physiological monitor using a lower weighted contribution of the heart rate variability relative to the heart rate than for the awake state. The system may further include a display device in communication with the second processor, the display device including a user interface configured to present a value to the user indicative of the stress score for the interval. The second processor may be disposed on one or more of: the wearable physiological monitor, the display device, and a remote server.

Dynamic Stress Scoring with a Model

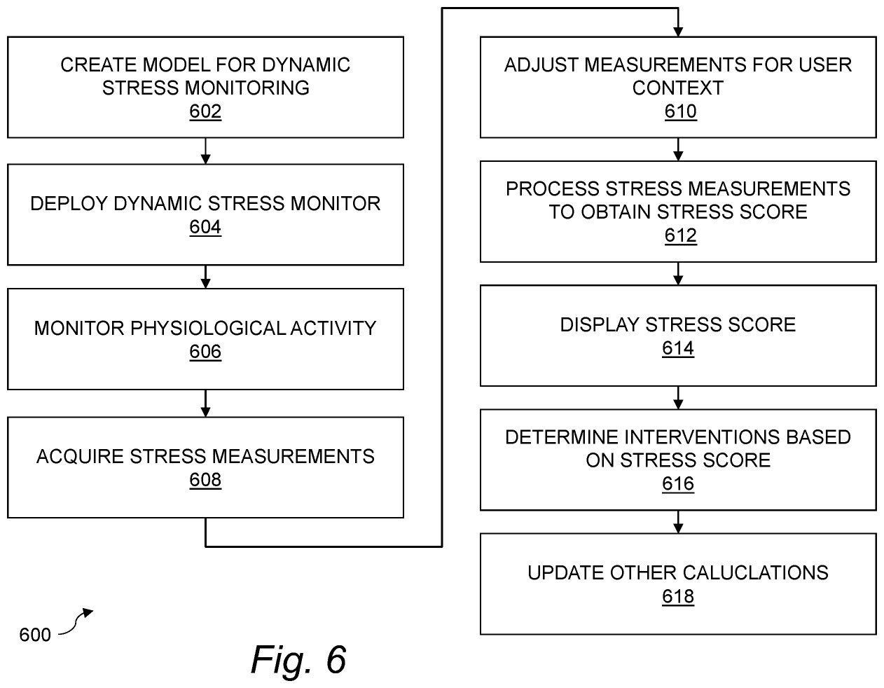

FIG. 6 is a flowchart of a method for dynamic stress monitoring. Dynamically monitored stress may include physiological stress, psychological stress, or some combination of these, which may be quantified and updated to provide current and cumulative measurements throughout a day, e.g., based on data obtained by a continuously wearable physiological monitor such as any as described herein. The method 600 can be used, for example, to provide recommendations for a user to perform basic interventions that can allow the user to adjust psychological stress stimuli and responses, adjust physiological stressors (e.g., exercise levels, work, physical discomfort, noise, physical exposures, etc.), and/or the like.

As shown in step 602, the method 600 may begin with creating a model for use in dynamic stress monitoring and scoring. In general, the model may include one or more machine learning models, statistical models, deterministic models, empirical models, analytical models, differential equations, linear equations, non-linear equations, transforms, formulas, regressions, rules, algorithms, data sets, and the like, which may be adapted alone or in combinations to create quantitative or qualitative scores for current stress based on physiological data and other user context.

In one aspect, the model may include a machine learning model, and creating the model may include training the machine learning model to report a quantitative or qualitative stress level based on, e.g., heart rate, heart rate variability, motion, blood pressure, respiration, and so forth. More generally, a machine learning model may be trained with any physiological signals or metrics that can be correlated with reasonable confidence to objective or subjective stress, and may be configured through such training to provide a quantitative or qualitative assessment of current stress based on corresponding inputs for user data, user context, and so forth.

The machine learning model may also or instead be trained using subjective stress data reported by and/or gathered from a user over the course of a day, or some other interval. For example, a user may be polled at some regular interval, say once per hour, and asked to report subjective stress. A data set may then be created based on the reported (or inferred) stress and physiological parameters such as heart rate, heart rate variability, motion, temperature (e.g., skin temperature), respiratory rate, skin conductance, blood pressure, and the like, some or all of which may be captured as instantaneous measurements or aggregate measurements (e.g., average measurements) over a preceding time window (e.g., thirty seconds, one minute, five minutes, etc.), or some combination of these. The training set of physiological measurements and stress labels may be used to create a model that predicts subjective stress based on one or more measured physiological parameters. More generally, subjectively reported stress may be used as a label or the like so that the machine learning model can be trained to generate a stress score reflecting data from physiological and/or environmental monitoring.

The machine learning model may also or instead be trained using a training set that includes a set of measured physiological responses to one or more predetermined stressors for a plurality of users. In this case, the training set may be labeled with inferred stress responses to the predetermined stressors, or the physiological data may be labeled with subjective stress scores reported by the users when exposed to a corresponding one of the predetermined stressors. That is, a group of users may be subjected to a number of stressors of varying intensity while measuring physiological parameters. These predetermined stressors may also represent activities selected to specifically induce or inhibit either mental or physical stress, for example by exposing users to various types and intensities of sensory inputs, physical tasks, mental tasks, and so forth. This can facilitate experimental control over the current stress imposed by the environment while measuring concurrent user responses.

By way of non-limiting examples, a user may be asked to perform activities known to increase blood pressure, engage the sympathetic or parasympathetic nervous system, or otherwise stimulate or inhibit an autonomic response. This may include activities such as isometric exercise, exposure to cold water, mental activities (e.g., mathematics or puzzle solving), or other activities known to stimulate an autonomic response. Other more specific measurements are known in the art for measuring an amount of response to stress, such as respiratory arrhythmia (changes in heart rate due to deep breathing), isometric hand gripping, cold pressor testing (immersion of hands or feet in cold water, e.g., 4 degrees Celsius), diving reflex (e.g., immersion of face in water), mental arithmetic, active standing (changes in heart rate during transition from supine to upright), and so forth. Objective scores may be associated with each activity based on a fixed estimate, or based on a concurrent measurement of heart rate, heart rate variability, blood pressure, or the like from each user while undergoing various activities. These numerical scores may be tagged with corresponding activities or stress scores and used as a data set of measured responses for training a machine learning model. The resulting, trained model may be used to predict a mental or physical stress based on measurements of physiological parameters such as those that can be readily captured from a wearable physiological monitor, and to provide an output a dynamic stress score such as a quantitative or qualitative stress score indicative of user stress in response to the measured physiological parameters.

The model may also or instead include an analytical model for formulaically calculating a stress score based on stress-related factors, or an empirical model derived from an analysis of subjective and/or objective stress criteria. For example, the model may include an empirically derived model that is configured to analytically (e.g., via a formula or algorithm) provide a stress score or other metric indicative of mental or physical stress based on physiological data obtained from a wearable physiological monitor. In general, a stress score may be calculated at a set time period—e.g., every minute or five minutes. Stress may be measured on a set scale—e.g., a scale from 0 to 3, where a score of 2-3 is high (meaning a user is likely excited, stressed, or highly activated), a score of 1-2 is medium (meaning a user is likely neutral, alert, or mildly activated), and a score of 0-1 is low (meaning a user is likely calm, relaxed, or sleeping).

The empirical model may use data from a predetermined time window (e.g., the immediate, previous one-minute or five-minute window) and may use a weighted combination of a heart rate variability (HRV) metric such as heart rate variability or a statistic derived or transformed therefrom, a heart rate (HR) metric such as a heart rate, an aggregate heart rate (e.g., an average heart rate), or a statistic derived or transformed therefrom, and a motion correction factor. Thus, in an aspect, an empirical model for calculating a dynamic stress score, S, may be based on the following equation (Eq. 1):

S=alpha*f(motion)*[w*(HR)+(1−w)*(HRV)] [Eq. 1]

The “HR” in the empirical model may be a score based on a user's measured heart rate, and may take into account user-specific, individualized data such as a user's personal baseline heart rate and maximum heart rate—e.g., using historical data, which can include historical baselines. For example, a user's heart rate from the previous five minutes may be mapped to a score between 0 and 3, taking into account the user's baseline resting heart rate and maximum heart rate. Other intervals may also or instead be used for this mapping, such as two minutes, one minute, or ten seconds. Shorter or longer intervals may also or instead be used provided they support a meaningful metric for calculating current stress. Thus, for example, a window of one hour would integrate a long history of physical and emotional responses, and would not typically reflect a current stress for a user. Similarly, a fraction of a second may not provide sufficient data for properly characterizing heart rate or heart rate variability, and/or may yield undesirable variability into a continuously reported metric.

A variety of mappings may be used to transform the measured value (e.g., heart rate) to a score that is scaled over the desired range. For example, a sigmoidal function or other function or distribution may be provided over an interval between a user's resting heart rate and maximum heart rate, and used to map a current measurement of heart rate to a heart rate score. For a sigmoidal function, if a user has a resting heart rate of 60 bpm and a maximum heart rate of 190 bpm, a heart rate of 100 may indicate that the user is relatively highly activated and the user may receive an HR score of, say, 2.1. The mapping may be linear, non-linear, lookup-based, and so forth, and the scale may be any range suitable for combination with other factors described above to provide an objective value for current stress. When a user's personal baseline heart rate is unavailable, e.g., because the user has just started using the device, or has a prolonged window of non-use or intermittent use, a baseline for a general population or for a similar cohort may be used temporarily as a substitute in order to facilitate dynamic stress score calculations.

“HRI” in the empirical model may be a score based on a user's measured heart rate variability, and may take into account historical user data such as a user's personal baseline HRV from a certain predetermined time period, such as an average for the preceding week, two weeks, three weeks, or the like. A current HRV measurement, such as an HRV for the previous minute, two minutes, or five minutes may be compared to the user's baseline HRV (or HRV distribution) distribution from the past 14 days, and the resulting value may be mapped to a score between 0 and 3 (where, again, these intervals are provided by way of example and where other intervals are also or instead possible). In one aspect, scores may be measured on a fixed scale, such as 0-3, where: a score of 0 to 1 represents a calm, relaxed, or sleep state; a score of 1 to 2 represents a neutral, alert, or mildly activated state; and a score of 2 to 3 represents an excited, stressed, or highly activated state.

It will be understood that “HRI” in the empirical model, and/or used in other stress score calculations included herein, may use any suitable quantitative measure of variations in heart rate. For example, HRV may use the root mean square of successive differences (RMSSD), which is a commonly used technique to assess heart rate variability (HRV). The RMSSD may be calculated, for example, by collecting heart rate interval data (e.g., RR intervals), calculating successive differences in RR intervals, squaring the differences, and calculating a root of the mean of these squared differences. In general, RMSSD is a measure of the short-term variability in heart rate and can be used as an indicator of parasympathetic nervous system activity. Higher RMSSD values generally indicate higher heart rate variability, which may be associated with better cardiovascular fitness and resilience to stress. Another measure that can be used to assess HRV involves examining the ratio of low-frequency (LF) to high-frequency (HF) components of heart beats (i.e., a LF/HF measure), where the LF and LF bands are derived from the power spectrum of heart rate variability. The LF/HF measure may be calculated by collecting heart rate interval data (e.g., RR intervals), performing a frequency domain analysis (e.g., using a Fourier transform or other spectral analysis technique) to decompose the HRV signal into different frequency bands (where frequency bands of interest are often categorized into LF (e.g., 0.04 to 0.15 Hz) and HF (e.g., 0.15 to 0.4 Hz)), calculating LF and HF power within the LF and HF frequency bands, and computing a LF/HF ratio. In general, the LF power reflects sympathetic and parasympathetic influences on the heart, and the HF power is primarily associated with parasympathetic activity. As such, the LF/HF ratio may be used as an index of sympathovagal balance, with higher values indicating a relative dominance of sympathetic activity, and lower values suggesting a greater influence of parasympathetic activity. Yet another measure that can be used to assess HRV is standard deviation of normal-to-normal (SDNN), which quantifies the overall variability in time between successive normal heartbeats (normal-to-normal, NN intervals or RR intervals) within a given time window, where a higher SDNN generally indicates greater HRV. The SDNN measure may be calculated by collecting a series of NN intervals over a time window, calculating an average NN interval over the time window, calculating the squared difference between each NN interval and the mean NN interval, calculating the variance by summing the squared differences, and computing the square root of the variance. Other HRV measures are also or instead possible.

The mapping may, for example, use a log-transformed 14-day history or distribution of HRV scores, identify a location of the current HRV within that distribution, and apply a 0 to 3 scaling function (e.g., linear, sigmoidal, look-up, or other) to obtain a final HRV score between 0 and 3. In one aspect, the mapping may be percentile based, e.g., by dividing an HRV distribution for the baseline HRV into percentiles, scaling these percentiles to a range of 0 to 3, locating the current HRV measurement within these percentiles, and assigning a score to the current HRV measurement based on the scale. This can include transforming HRV data for the baseline profile into a more normal distribution, e.g., using a log transform or the like. For example, if a user's HRV is typically between 43 milliseconds (ms) and 134 ms, and the HRV from the previous five minutes is 67 ms (and assuming 67 ms falls within a relatively high percentile within the 14 day distribution), that would indicate a high level of activation and the user may receive an HRV score of 2.6. As with the transformed HR score described above, the HRV score may be mapped using any suitable linear, non-linear, lookup-based, or other model, and the scale may be any range suitable for combination with the other factors described above to provide an objective value for the current stress.

When a user's individualized HRV baseline distribution is unavailable or unknown, a default baseline may be used by the empirical model, such as a default HRV baseline based on a population of users. After a certain number of values of HRV data are obtained for a particular user (e.g., >1000 values or >15 minutes of HRV data), the default HRV baseline may be changed to a subjective HRV baseline for the user. In another aspect, the personalized HRV baseline may be deferred until at least one or two full days of HRV data are available.

The empirical model may weight between contributions of heart rate and heart rate variability using a weight, “w′.” In one aspect, the weight may vary based on the current value of the HR and HRV scores, or based on other user context. For example, when HR is low (as it tends to be during sleep), HR may be weighted more heavily to ensure that stress scores stay low during sleep. By way of further example, when the HR is above a certain threshold, e.g., 100 bpm, this may indicate that a user is likely awake, and the HR and HRV scores may be weighted equally (e.g., a weight of 0.5) so that the stress score reflects a combination of heart rate and heart rate variability. Also or instead, the weights may be dynamically adjusted based on a confidence in the readings. By way of example, the weight given to HRV may be reduced when the quality of the data does not support a reliable HRV reading.

A motion score, “f(motion),” may be used in the empirical model as a proxy for physical activity, and may use data from accelerometers, gyroscopes, and/or other motion sensors or the like—e.g., disposed on a wearable physiological monitor—to measure motion and adjust the dynamic stress score accordingly. Motion data from one or more of these sensors may, for example, be converted into a motion score and used in turn to weight the stress score in a manner that reflects the user's degree of physical motion. By way of example, if a user is moving, the overall score may be decreased to account for contributions of physical activity to corresponding changes in HR and HRV, while if a user is motionless, the motion score may be at or near 1 to reflect that any elevated cardiac activity is likely a result of non-physical stress. Thus, if a user is sitting idly, the motion score may be about 1, say 0.98, so that the stress score responds fully to changes to HR and HRV caused by non-physical demands and stressors. More generally, the motion score may be any function, equation, constant, variable, or combination of these suitable for transforming motion data into a value (or values) for use in adjusting the calculated stress score based on sensed motion data for the user.

In general, the calculated stress score may be scaled by a fixed or variable scaling factor, alpha. This scaling factor may be used to conform the range of possible calculated scores to a desired reporting range for presenting the stress score (e.g., 0-3) to a user or using the stress score in subsequent calculations. In this manner, alpha may assume any value or range of values suitable for the underlying data and calculations.

While the foregoing example provides a useful formula for relating motion, heart rate, and heart rate variability to a current stress being experienced by a user, it will be understood that other formulas, algorithms, and/or other mathematical models may also or instead be used to calculate an estimated current stress of a user in a manner that permits dynamic, real time, and/or intraday monitoring and reporting of a dynamic stress score. For example, a simplified model may rely exclusively on motion and heart rate to objectively calculate stress. Or, a more complex model may incorporate respiratory rate, skin temperature, hydration status, blood pressure, galvanic skin response, and so forth. Still more generally, any analytical model, machine learning model, empirical model, statistical model, or other model or analytical framework, or combination of these, may be used to estimate a dynamic stress level of a user for purposes of intraday monitoring, coaching, reporting, and the like.

As shown in step 604, the method 600 may include providing the model for use, e.g., by deploying the model in a dynamic stress monitor, which may include hardware, software, or some combination of these configured to apply the model to acquired data in order to generate stress scores indicative of current stress for a user. As a significant advantage, a machine learning model may be compressed for local deployment on a wearable physiological monitor, or some other local user device such as a smartphone, tablet, or laptop, to facilitate local processing of continuous updates without the need for a server. The machine learning model may also or instead be deployed at a server or other remote processing resource, which permits the use of larger, more refined machine learning models, but may introduce significant latency into the user experience at times when a real time estimate of stress might be desired. Other models, such as those described herein, may also or instead be adapted for deployment on a wearable physiological monitor, or for distributed deployment between a wearable device, a user's personal computing device, and/or a remote server, e.g., using the techniques described herein.

In general, deployment of the model may include storing the model, e.g., as parameters, data sets, weights, rules, instructions, and so forth, in a memory of a device. Where the model is to be locally deployed on a wearable monitor, this may include storing the model in a memory of the wearable monitor. In another aspect, the model may be stored in a memory of a user device such as a smartphone, tablet, or laptop, which may receive data from the wearable monitor and apply the received data to the model stored in local memory. In another aspect, the physiological monitoring data from the wearable device may be transmitted to a remote server that stores the model for execution against acquired data at a remote location.

As shown in step 606, the method 600 may include monitoring physiological activity with a wearable physiological monitor, such as any of the monitors described herein. This may include acquiring physiological data such as heart rate data, heart rate variability data, motion data, body temperature data, respiratory rate data, skin temperature data, skin conductance data, blood pressure data, and so forth. In general, this physiological data (also referred to herein as “physiological parameters”) may be transmitted to a remote resource for calculation of daily metrics such as strain, sleep, and recovery as described herein.

In one aspect, monitoring physiological activity may include providing a heart rate variability metric for a user who is wearing a physiological monitor. In general, providing the heart rate variability metric may include measuring or calculating the metric, or receiving the metric from another source, and/or storing the metric in a memory. The heart rate variability metric may be a measured heart rate variability, a calculated heart rate variability, a raw number related to heart rate variability, an aggregate for heart rate variability over an interval, an output of a calculation that includes heart rate variability as an input, and the like. In certain aspects, providing the heart rate variability metric includes acquiring the heart rate variability metric directly or indirectly from a physiological monitor worn by the user. By way of example, providing the heart rate variability metric may include acquiring an aggregate heart rate variability measurement over an interval with a wearable physiological monitor, and/or calculating or deriving a heart rate variability metric based on physiological data from the monitor using any of the techniques described herein.

Monitoring physiological activity may also or instead include providing a heart rate metric for the user. In general, providing the heart rate metric may include measuring or calculating the metric, or receiving the metric for another source, and/or storing the metric in a memory. The heart rate metric may be a measured heart rate, a calculated heart rate, a raw number related to heart rate, an aggregate or baseline heart rate over an interval, an output of a calculation that includes heart rate as an input, an HRV or HRV related metric, and the like. In certain aspects, providing the heart rate metric includes acquiring the heart rate metric, directly or indirectly, from a physiological monitor worn by the user. By way of example, providing the heart rate metric may include acquiring an aggregate heart rate measurement over an interval with a wearable physiological monitor, and/or calculating or deriving a heart rate metric based on physiological data from the monitor using any of the techniques and devices described herein.

As shown in step 608, the method 600 may include acquiring a plurality of stress measurements for a user, e.g., by processing the physiological data received from a wearable monitor using the techniques described herein to generate corresponding stress scores. For example, this may include acquiring the stress measurements by applying the model to physiological data acquired over any suitable interval for which stress might be evaluated, such as one minute, two minutes, five minutes, ten minutes, thirty minutes, or sixty minutes. The frequency of measurements may be any suitable frequency consistent with the capabilities of the computing platform executing the dynamic stress monitor. For example, the stress level may be calculated once every second, every ten seconds, every thirty seconds, every sixty seconds, and so forth. It will be understood that the greater the measurement frequency desired, or the smaller the latency in reporting stress, the more advantageous it may be to locally deploy the dynamic stress monitor on the wearable monitor or another device locally coupled to the wearable monitor and available to a user.

The interval over which measurements are taken may also vary. In one aspect, the interval may usefully be in a range between three minutes and ten minutes, which provides a sufficient interval to avoid spurious estimates of stress while also excluding historical data that might not be directly relevant to an individual's current stress level. Longer or shorter intervals may also or instead be employed, and the method 600 may also include reporting stress levels for multiple intervals concurrently, e.g., by graphically displaying a time series of stress scores for overlapping or non-overlapping intervals, or by displaying a short term and long term stress score (e.g., ten seconds and ten minutes).

In general, any of the techniques described herein for calculating a stress score or stress metric may be used to calculate an instantaneous stress measurement for a set of physiological data from a monitor. Acquiring a stress measurement may also or instead include scaling resulting calculated values with a function or algorithm that transforms the stress estimate into a value within a predetermined range. For example, the stress estimate can be scaled to a range of 0-3, 0-10, or some other range that provides a useful indicator of current stress for the user. In one aspect, scaling may include binning each stress measurement into an integer value or the like. In one aspect, scaling may also or instead include applying a non-linear scaling that transforms a majority of the stress estimates for an individual to a lowest value for the stress score range, e.g., so that some predetermined percentage of lowest measurements receive a scaled value of 0 or 1. This algorithmically captures the notion that, during most of the day, a user will likely not be experiencing significant stress that would require intervention or remediation, or that might have a negative cumulative effect on readiness for strain.

As shown in step 610, the method 600 may include adjusting stress measurements for a user context. For example, this may include determining a resting state of the user, which provides a context useful for properly interpreting cardiac metrics, e.g., where a relatively high or low value for a metric may have different meanings depending on the user's resting state. This may also or instead include determining a sleep state, automatically detecting an activity type, or otherwise detecting or evaluating the user's context to better interpret physiological data acquired from the physiological monitor.

In one aspect, this may include adjusting stress measurements according to circadian rhythm. Components of the stress score, such as HR (especially when motionless) and HRV, may vary significantly and predictably over the course of a day based on an individual's circadian rhythm, and more specifically where in the cyclical circadian rhythm a user is when the physiological data is acquired. Cardiac metrics may have significantly different patterns and average values shortly before sleep, when sleeping, shortly after waking, and around mid-day. Therefore, these measures may be normalized for a user relative to the time of day when using these metrics as a baseline or measurement to detect stress, e.g., to more accurately detect deviations in a calculated current stress score that are specifically related to stressors, as distinguished from circadian effects. This approach advantageously supports continuous or periodic stress monitoring more accurately outside of a clinical setting, and supports more effective coaching and interventions. It will be noted that these circadian adjustments may be performed on raw data acquired, e.g., in step 606 above, where the received or calculated values for HR, HRV, and so forth can be directly calibrated to the circadian cycle, or these adjustments can be performed after calculating stress measurements according to the effect of changes in the raw metrics on calculated stress measurements. In either case, the resulting stress score can be better calibrated to an individual's current circadian state. In one aspect, the circadian rhythm may be detected or inferred for a user based on motion, activity, sleep, cardiac activity, and so forth. In another aspect, the circadian rhythm may be inferred based on clock time or other data.

For a population of users, heart rate has been shown to be elevated (relative to a daily average) during the daytime and depressed leading up to and throughout the night, whereas heart rate variability increases throughout sleep, peaks in the early morning, and subsequently decreases throughout the day. Because heart rate and heart rate variability can exhibit cycles corresponding to the circadian rhythm, measures (such as current stress) that rely on these cardiovascular parameters may be corrected for the time of day when the measurements were taken. Similarly, daily measurements of, e.g., a representative resting heart rate or heart rate variability may usefully be captured each day at a particular point in the circadian rhythm (e.g., shortly before waking, shortly after waking, at some particular period or stage of sleep, etc.) to facilitate consistency of measurements and accuracy of resulting calculations.

Thus, in one aspect, a resting state for the user may be determined based on a circadian state of the user. For example, sleep and waking cycles for a user may be detected based on data from a wearable monitor and a current circadian state may be evaluated and expressed, e.g., as a probability of a sleep state for the user, a detected sleep state for the user, and so forth. For example, in one aspect a circadian rhythm model for a population (e.g., a population of users that have similar characteristics to the user) or for the particular user, may be created and used to detect a sleep state based on patterns in, e.g., respiratory rate, heart rate, motion, skin temperature, and so forth. Where a circadian state is used as a proxy for the resting state, the circadian state may be expressed as discrete states, e.g., an asleep state or an awake state. Further, the asleep state may include one or more sub-states for one or more corresponding stages of sleep. To this end, the resting state may be determined with a sleep detection algorithm that evaluates physiological metrics and/or motion to determine a sleep state for the user. As described herein, when calculating a stress score using, e.g., Eq. 1 described above, a weight (e.g., w) for a heart rate metric may increase when the resting state is an asleep state for the user, and/or a complementary weight (e.g., 1−w) for the heart rate variability metric may decrease when the resting state is an asleep state for the user. In another aspect, the circadian state may be expressed as a probability (e.g., 90% likelihood of sleeping), or the circadian state may be expressed as an estimated location within a twenty four hour circadian cycle, any of which may be used to variably weight between different physiological metrics, or otherwise adjust a dynamic stress score to better reflect a user's stress based on the user's context.

In another aspect, the dynamic stress score may be adjusted based on a user's resting state. The resting state, which characterizes a general state of activity rather than, e.g., a sleep state or circadian status, may be evaluated based on a difference between the current heart rate of the user (or other suitable heart rate metric) and a resting heart rate of the user. For example, the weighted combination described above in Eq. 1 may use a first weight for a first component of the stress score based on the heart rate metric, where the first weight is based on the resting state of the user, and the weighted combination may use a second weight for a second component of the stress score based on the heart rate variability. In general, the first weight may increase as the difference decreases, e.g., as the current or average heart rate approaches a resting heart rate for the user. For example, the first weight may approach 1 as the heart rate metric approaches a resting heart rate for the user. The first weight may monotonically approach 1 as the heart rate metric approaches the resting heart rate, or the first weight may sigmoidally approach 1 as the heart rate metric approaches the resting heart rate. In another aspect, the second weight may decrease as the current or average heart rate approaches a resting heart rate for the user. The adjustment may include using a first weight of w and a second weight of 1-w as described above, or the adjustment may include independently controlling the manner in which the second weight decreases, e.g., to a value between 0.5 and 0, as the heart rate metric approaches the resting heart rate.

The resting heart rate used to evaluate a resting state may, for example, be automatically measured using any of the techniques described herein, and may include a multi-day average, an average of several measurements during a sleep cycle for a user, or some combination of these, or any other suitable technique. In this context, the current heart rate or heart rate metric may also be calculated as a moving average, windowed average, or other metric that tends to smooth heart rate over a local time interval (e.g., of a few seconds or minutes) in order to reduce large, instantaneous changes in the resulting, calculated dynamic stress score.

In another aspect, the distance between the current or aggregate heart rate (e.g., average heart rate) and the resting heart rate may be characterized by bins, ranges, or other general categories to facilitate local continuity in calculated values, e.g., from measurement to measurement over the course of a few minutes or an hour. Thus, in one aspect, determining a distance may include determining whether a heart rate metric for an interval is within a predetermined range of a resting heart rate for the user. For example, in response to determining that the heart rate metric is outside of a predetermined range of a resting heart rate for the user, the method 600 may include calculating a stress score for the user over the interval based on a first weighted combination of plurality of measurements of a heart rate and a heart rate variability acquired during the interval from the physiological monitor. And, in response to determining that the aggregate heart rate is within the predetermined range of a resting heart rate for the user, the method 600 may include calculating the stress score for the user based on the plurality of measurements using a second weighted contribution of the heart rate variability (more specifically, a lower weighted contribution of the heart rate variability) relative to the heart rate than in the first weighted combination used when it is determined that the aggregate heart rate is outside the predetermined range.

More generally, adjusting the dynamic stress score for a user context may include, in response to determining that a heart rate metric such as an aggregate heart rate (e.g., average heart rate) for the user is outside a predetermined range from a resting heart rate for the user, applying a first algorithm to calculate the stress score for the user, and, in response to determining that the heart rate metric is within the predetermined range from a resting heart rate for the user, applying a second algorithm to calculate the stress score for the user. While the second algorithm may include a formula using a lower weighted contribution of the heart rate variability relative to the first algorithm, it will be understood that the second algorithm may more generally include any formula, algorithm, technique or the like suitable for evaluating user stress when the current heart rate is at or near the resting heart rate for the user.

In another aspect, the resting state may also or instead be a discrete resting state selected based on one or more predetermined ranges for the user's heart rate, e.g., relative to a resting heart rate or some other suitable cardiac benchmark.

As illustrated in Eq. 1, calculating the stress score may also or instead account for motion data acquired during the interval from the physiological monitor or another device. In general, user activity may provide context useful for adjusting stress measurement calculations. For example, high-intensity physical stressors such as exercise can elevate HR and reduce HRV for many hours after the physical stressor has been removed due to persistent activation of the sympathetic nervous system and inhibition of the parasympathetic nervous system. Conversely, low-intensity exercise has been shown to reduce heart rate for up to twenty four hours following an exercise, possibly due to an acute reduction in total and regional vascular resistance. These activities can be detected based on user movement, and the corresponding physiological patterns can be used to shape expectations for HRV and HR when calculating a current stress, e.g., using any of the models described herein. In another aspect, these patterns may be measured and characterized for an individual in order to better identify local stressors unrelated to physical activity, circadian rhythm, and so forth.

As shown in step 612, the method 600 may include processing the plurality of stress measurements over an interval to provide a dynamic stress score that can be reported to a user for the interval. This may, for example, include averaging, summing, or otherwise aggregating individual stress measurements into a representative value for the interval of interest. As with individual stress measurements, processing the plurality of measurements for the interval may include scaling the resulting stress score with a function or algorithm that transforms the aggregated or dynamic stress score into a value within a predetermined range. For example, the stress score may be scaled to a range of 0-3, 0-10, or some other range that provides a useful indicator of current stress for the user. In one aspect, the scaling may include binning the stress score into an integer value or the like. Scaling may also or instead include applying a non-linear scaling that transforms a majority of the dynamic stress scores for an individual to a lowest possible value for the dynamic stress value. This algorithmically captures the notion that, during most of the day, a user will likely not be experiencing significant stress that might require intervention or have a negative cumulative effect on readiness for strain.

As also described herein, a stress score may be formulated to report or distinguish between mental stress (e.g., from emotional, intellectual, or other psychological stress) and physical stress (e.g., from physical activity, exposure, and so forth), and to inform a user of the degree to which either or both of these components are contributing to a current dynamic stress. For example, while individual stress scores are generally described, the dynamic stress score may also or instead be reported as two or more different scores that separately evaluate mental stress, physical stress, composite stress, and so forth. To measure these different stressors independently, physiological signals such as heart rate and heart rate variability data may be individually weighted and combined to estimate overall physiological stress, and then motion data may be incorporated to isolate physically derived stress from other types of stress. In another aspect, a difference between a measured heart rate and a predicted heart rate (e.g., based on motion data) may be used to derive a score that reflects mental stress. More generally, any techniques for isolating and/or identifying the impact of different sources of stress may be used to provide a multi-metric stress score for a user.

As shown in step 614, the method 600 may include presenting the dynamic stress score or other derived metric for near term stress to the user in a display. This may, for example, include presenting the dynamic stress score on a display of the wearable physiological monitor, a display on some other user device such as a smartphone or tablet, or in any other suitable user interface. This may also or instead include tactile feedback such as haptic feedback provided by a haptic device in the wearable monitor, audio feedback, or other feedback that functions independently from, or in cooperation with, a display on the wearable physiological monitor. As described herein, the dynamic stress score may be evaluated using a combination of processing resources on a wearable physiological monitor, a user's computing device(s) (e.g., smartphone, tablet, laptop, and so forth), a server, or other computing device(s), as well as combinations of the foregoing. As such, the score may be provided from, and/or rendered on, any suitable computing device or combination of computing devices and display systems useful for communicating the dynamic stress core to the user. Displaying the dynamic stress score may also or instead include displaying a multi-metric stress score (e.g., for physical and mental stress components, or for different activities or time windows), a time series of dynamic stress scores (e.g., as a timeline or similar graph), a chart of recent scores, an average of recent scores, and so forth.

As shown in step 616, the method 600 may include determining interventions based on the dynamic stress value, and/or presenting suggested interventions to the user. In general, this may include a variety of coaching recommendations, lifestyle suggestions, short term stress management exercises, and so forth. In one aspect, this may include determining suitable thresholds for intervention, e.g., based on typical stress score ranges for a user or for a population, based on explicit user preferences, or based on other factors, as well as combinations of these. For example, this may include identifying a threshold for the dynamic stress score that is indicative of acute stress. This may also or instead include identifying a threshold indicative of autonomic activation. In general, interventions may include alerts such as a text, email, audible alert, haptic alert, or the like. The wearable monitor may provide this alert where it has suitable user interface capabilities, or the alert may be provided by some other user device such as a smartphone, smart watch, or the like. The intervention may include recommended remediations for remediating stress, e.g., general suggestions to stop or avoid stressful activities, or specific recommendations such as stopping a current workout, engaging in wellness exercises such as deep breathing or other breath work, meditation, or the like, or consulting with a health care provider. This can also or instead include recommendations to increase alertness depending on the needs of the user, along with interventions that can provide an increase in engagement and/or alertness. These recommendations may be provided in real time, that is, effectively immediately upon detection of the acute stress, with no observable latency to the user.

It will be understood that dynamic stress monitoring as described herein may include adjusting a stress score and/or stress alerts depending on whether the stress stems from physical stress or mental stress, and stress scoring and interventions may include an analysis for distinguishing between these components of stress. By way of example, sources of stress can be determined using motion data or the like as described herein, e.g., to estimate if a user is physically active when under stress, or to otherwise isolate physical and psychological contributors to elevated stress. Such a determination may also or instead use input directly from a user to determine if the user is exercising (e.g., if the user manually provides input indicating that they are performing an activity), or to determine if the user is reporting subjective stress. Such a determination may also or instead be based on a user state, e.g., for activity type, sleep state, and so forth, any of which may usefully be autodetected by the wearable physiological monitor, reported by the user, or some combination of these.

Coaching may also or instead include interventions during physical activity, e.g., by recommending pausing or terminating physical activity where the current stress appears disproportionately high for a current level of physical activity, or by recommending additional and/or alternative exercise where the current stress appears disproportionately low for the current level of physical activity. More generally, any apparent mismatch between measured stress and physical activity may be communicated to the user in an alert, and/or used as the basis for coaching or activity recommendations targeting the mismatch.

As shown in step 618, the method 600 may include updating other calculations. For example, stress may be aggregated over the course of a day, and may be used to revise other user metrics. In one aspect, a recovery score for the user—indicative of readiness to perform physical activity—may be refined based on aggregated dynamic stress by reducing the user's recovery score for a current day as stress accumulates, or by making intra-day refinements to coaching recommendations, e.g., to reduce the recommended physical activity for a day as stress accumulates. In another aspect, accumulated stress, such as stress that is objectively measured with a dynamic stress score, but that does not appear to be attributable to physical activity, may be added to a user's measurement of strain for the day, e.g., so that sleep and recovery metrics for a next daily cycle can be adjusted accordingly, and/or so that coaching recommendations such as a suggested amount of sleep can be adapted to account for the increased stress detected for the user.

It will be understood that the displays, visualizations, notifications, coaching recommendations and the like described with reference to FIG. 6 may also or instead be used in combination with any of the other stress scoring methods and systems described herein.

According to the foregoing, in an aspect, a method described herein includes: providing a heart rate variability metric for a user; providing a heart rate metric for the user; determining a resting state of the user; and calculating a stress score for the user. The stress score may be calculated based on a weighted combination of the heart rate variability metric and the heart rate metric, the weighted combination using a first weight for the heart rate metric based on the resting state of the user, and the weighted combination using a second weight for the heart rate variability based on the resting state of the user. The method may be embodied in a computer program product including computer executable code stored in a non-transitory computer readable medium that, when executing on one or more computing devices, performs the corresponding steps.

In another aspect, a method described herein includes: measuring an aggregate heart rate (e.g., average heart rate) of a user over an interval with a physiological monitor; determining whether the aggregate heart rate over the interval is within a predetermined range of a resting heart rate for the user; in response to determining that the aggregate heart rate is outside the predetermined range, calculating a stress score for the user over the interval based on a plurality of measurements of each of a heart rate, a heart rate variability, and a motion acquired during the interval from the physiological monitor; in response to determining that the aggregate heart rate is within the predetermined range, calculating the stress score for the user based on the plurality of measurements using a lower weighted contribution of the heart rate variability relative to the heart rate than a weighted contribution used when it is determined that the aggregate heart rate is outside the predetermined range; and displaying a value to the user indicative of the stress score for the interval. The method may be embodied in a computer program product including computer executable code stored in a non-transitory computer readable medium that, when executing on one or more computing devices, performs the corresponding steps.

In another aspect, a method described herein may include determining a sleep state of a user; in response to determining that the sleep state is an awake state, calculating a stress score for the user over an interval based on a plurality of measurements of each of a heart rate, a heart rate variability, and a motion acquired during the interval from a physiological monitor worn by the user; in response to determining that the sleep state is an asleep state, calculating the stress score for the user based on the plurality of measurements using a lower weighted contribution of the heart rate variability relative to the heart rate than for the awake state; and displaying a value to the user indicative of the stress score for the interval. The method may be embodied in a computer program product including computer executable code stored in a non-transitory computer readable medium that, when executing on one or more computing devices, performs the corresponding steps.

Dynamic Stress Scoring with Probability Distributions

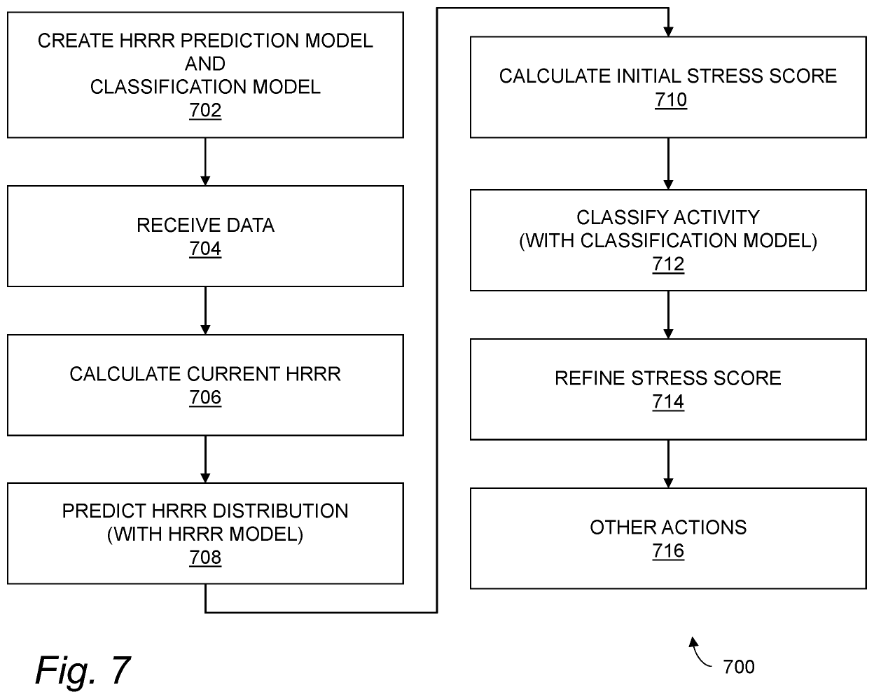

FIG. 7 is a flowchart of a method for calculating a stress score. In general, a model may be trained to generate a probability distribution for an expected heart rate metric and/or other physiological metric (e.g., electrodermal measurement/activity, blood pressure, respiratory rate, skin temperature, body temperature, and the like) based on a user's context, and a stress score may then be evaluated by comparing a currently measured heart rate metric to a predicted probability distribution for the metric under current conditions. As with the other models described herein, this model may generally be balanced to emphasize psychological stress (e.g., by using motion artifacts to reduce the calculated stress) or physical stress (e.g., by using heart rate metrics independently from measured user motion), and/or to account for the effects of periods of sleep and/or the circadian rhythm on current stress.

As shown in step 702, the method 700 may begin with creating a first model to generate a probability distribution of expected heart rate reserve ratios and a second model to identify an activity type.

In general, the first model may be a heart rate reserve ratio prediction model trained to predict a heart rate reserve ratio (“HRR ratio,” or “HRRR”) for the user based on other inputs, e.g., from a physiological monitor worn by the user. For example, the model may be trained to predict an HRR ratio for the current heart rate based on inputs such as motion data characterizing user movement, e.g., based on data from an inertial measurement unit, gyroscope, and/or other motion sensing system(s) in the physiological monitor. The model may also or instead be trained based on features extracted from these or other raw data feeds from the physiological monitor. For example, one or more features may be derived from motion data based on aggregations over one or more different time windows, e.g., for windows over the past 30, 60, and/or 90 minutes, and may include summary statistics such as a median, a mean, a maximum, a minimum, a standard deviation, an autocorrelation, and so forth. Where available, other motion data such as GPS data may also or instead be used as training data. More generally, any features, attributes, derived quantities, or the like either from individual measurements or a time series of measurements, may be used as engineered features, and/or may be used as training data for a machine learning model to predict an HRR ratio distribution, particularly where the underlying data is demonstrably correlated to the HRR ratio for a user. It will be understood that other data may also or instead be used to train the model, such as user attributes and/or features engineered or derived therefrom. This data may also be used to scale model inputs and/or otherwise adapt model results to a particular user or user type. For example, user attributes such as age, height, weight, gender, and the like may assist in correctly modeling an expected HRR ratio and creating an accurate machine learning model to predict an HRR ratio based on other inputs such as time series data from a physiological monitor.

In general, a model may be trained for these inputs using a result or labeled data set based on corresponding HRR ratio data for users. It will be understood that, while the following description discusses the HRR ratio for a particular user, the machine learning model may more generally be trained for a population, a subset of a population, a particular user, or some combination of these (such as by training a large model based on population data, and then refining or otherwise adapting the model with data for a particular user). Furthermore, while a probability distribution for HRRR is described, other heart rate metrics (e.g., HRV) and/or other physiological metrics (e.g., electrodermal, blood pressure, respiratory rate, skin temperature, body temperature, and the like) may also or instead be used.

In general, the HRR measures the range of expected heart rates, generally expressed as a difference between the maximum heart rate and the resting heart rate. For purposes of evaluating a user's current stress level and for training the machine learning model, a heart rate reserve ratio (HRRR) may be used as a target metric, which expresses the current heart rate relative to the HRR as follows:

$$H R R R = \frac{H {R}_{c u r} - H {R}_{b a s e}}{H {R}_{max} - H {R}_{r e s t}} \qquad [ Eq . 2 ]$$

where
   HR_{cur} is the current heart rate for a user, in beats per minute (bpm),
   HR_{base} is a baseline resting heart rate for the user, in bpm,
   HRₘₐₓ is an estimate of the maximum heart rate for the user, in bpm, and
   HRᵣₑₛₜ is a resting heart rate for the user, in bpm.

The current heart rate for the user (HR_{cur}) may be the user's current heart rate, and may be measured in a number of ways. For example, this may include an instantaneous measurement of current heart rate. Because this metric may be highly variable, the instantaneous heart rate may be windowed, averaged, or otherwise processed to obtain a more consistent measure of current exertion. For example, the current heart rate may be a moving average of the heart rate over a local interval such as ten seconds, one minute, five minutes, or the like.

The baseline resting heart rate (HR_{base}) may represent a basic, sedentary heart rate for the user, and may be measured in a variety of ways. For example, a resting heart rate is conventionally measured shortly after waking, and a measurement may be automatically or manually captured at this time, either for the current day, for a prior day, or for a history of preceding days. In another aspect, the baseline resting heart rate may be measured, e.g., for the current day, based on one or more recent periods of inactivity during which a relatively low, stable heart rate was observed. In another aspect, the resting heart rate may be measured during sleep, e.g., during a particular phase of sleep such as during deep sleep, or more specifically during the last phase of deep sleep before waking. For a resting heart rate measured during sleep, the heart rate may be measured multiple times each night (e.g., for several sequential deep sleep cycles), with the cycles selected based on order (e.g., the last two or three deep sleep cycles before waking) or data quality (e.g., the deep sleep cycles having the highest quality heart rate data acquisition). The resting heart rate may also or instead be measured over the course of several days in order to provide an aggregate resting heart rate (e.g., average resting heart rate) and to avoid local variations in individual measurements and/or idiosyncratic cardiac activity that might not be indicative of a user's true resting heart rate. For example, the baseline resting heart rate may be a derived statistic based on a median or average resting heart rate measurement for the immediately preceding fourteen days, or some other window of time. In another aspect, a user may manually measure and enter a resting heart rate for use in HRRR calculations.

The maximum heart rate (HRₘₐₓ) may represent an estimated peak or maximum heart rate for the user, and may be measured in a number of ways. For example, one conventional measure of maximum heart rate is calculated by subtracting a user's age from 220. For example, a 50 year old would have an estimated maximum heart rate of 170 beats per minute. An estimated maximum heart rate may also be adjusted, e.g., according to gender, weight, height, and so forth. In another aspect, historical heart rate data for the user may be used to calculate a maximum heart rate, e.g., based on a history of exercise activity over some window of time. In another aspect, historical heart rate data may be used to adjust an age-based estimate of maximum heart rate, e.g., by adjusting the estimate based on a history of peak heart rates—e.g., during detected exercise activity—that are consistently above or below the estimated maximum heart rate.

The resting heart rate (HRᵣₑₛₜ) may represent a heart rate while at rest. As with the baseline heart rate, the resting heart rate may be measured shortly after waking, and a measurement may be automatically or manually captured at this time, either for the current day, for a prior day, or for a history of preceding days. In another aspect, the resting heart rate may be measured, e.g., for the current day, based on one or more recent periods of inactivity during which a relatively low, stable heart rate was observed. In another aspect, the resting heart rate may be measured during sleep, e.g., during a particular phase of sleep such as during deep sleep, or more specifically during the last phase of deep sleep before waking. For a resting heart rate measured during sleep, the heart rate may be measured multiple times each night (e.g., for several sequential deep sleep cycles), with the cycles selected based on order (e.g., the last two or three deep sleep cycles before waking) or data quality (e.g., the deep sleep cycles having the highest quality heart rate data acquisition). The resting heart rate may also or instead be measured over the course of several days in order to provide an aggregate resting heart rate (e.g., average resting heart rate) and to avoid local variations in individual measurements and/or idiosyncratic cardiac activity that might not be indicative of a user's true resting heart rate. For example, the resting heart rate may be a derived statistic based on a medium resting heart rate measurement for the immediately preceding fourteen days, or some other window of time.

In one aspect, the resting heart rate (HRᵣₑₛₜ) may be the same metric or derived metric as the baseline resting heart rate (HR_{base}), which conveniently normalizes the heart rate reserve ratio to exactly one when the current heart rate is equal to the maximum heart rate. In another aspect, these numbers may be individually calculated based on different reference points, e.g., in order to incorporate both long term and short term heart rate trends into a heart rate reserve ratio calculation. For example, the baseline heart rate may be calculated based on recent data, e.g., a heart rate shortly after waking on the current day, or an average of the heart rate shortly after waking on one or two recent days. This facilitates a comparison of the current heart rate to a baseline resting heart rate for the day that the heart rate reserve ratio is being calculated. The resting heart rate, on the other hand, may be calculated as a moving average of a representative heart rate over a longer period of time, e.g., several days or a week, and may be based on heart rate measurements during a sedentary period, e.g., during one or more intervals of deep sleep from one or more preceding days. This may also or instead include a weighted average that weights recent measurements more heavily than prior measurements, and/or may include a window that extends over several weeks or more, e.g., in order to better track general fitness rather than a potentially highly variable single day measurement.

More generally, it should be understood that the heart rate reserve, the heart rate reserve ratio, and each of the constituent metrics, may be measured or calculated in a number of different ways. While metrics such as the heart rate reserve are well known in the art and provide a useful benchmark for a personalized measure of cardiovascular activity or effort, any other heart rate metric that objectively evaluates a current heart relative to a range of possible or expected heart rates for the user may be used in addition to or instead of the heart rate reserve ratio (HRRR) for purposes of training a machine learning model and/or evaluating a user's current level of cardiac activity, e.g., for calculating a stress score as described herein. Also or instead, other physiological metrics may be used (e.g., electrodermal, blood pressure, respiratory rate, skin temperature, body temperature, and the like), i.e., in addition to or instead of heart rate metrics.

With the target metric calculated over the temporal range of training data (e.g., motion data acquired from accelerometers, gyroscopes, etc.), heart rate data, engineered features, user data, etc., a machine learning model may be trained to predict HRRR for a user based on corresponding inputs. In one aspect, an HRRR prediction model may be trained to predict a probability distribution for HRRR expressed, e.g., as a set of quantiles characterizing the distribution of expected HRRR values corresponding to the features of the training data. As a significant advantage, this permits scoring of current data for a user relative to a range of likely results, and scales the result within a probability distribution that is contextualized according to current user data. Thus, for example, when a user is in motion-say, running or walking-a distribution of expected HRRRs will reflect a correspondingly elevated heart rate (and HRRR). The same elevated heart rate, while a user is at rest, may indicate greater stress, e.g., emotional stress, which can be captured by comparison of the calculated HRRR for the user to the probability distribution corresponding to motion data for the user that shows a lack of physical activity.

The probability distribution may be expressed at any suitable level of granularity for subsequent comparisons to actual HRRR data for the user. In general, any quantization may be used for this distribution. For example, the quantiles may be linearly distributed over the range of possible values, e.g., as quartiles, deciles, or the like. For example, for deciles, the first decile may be the 10th percentile at a number below which 10 percent of HRRRs are expected to fall, a 20th percentile at a number below which 20 percent of HRRRs are expected to fall, and so forth. In another aspect, the distribution may be non-linearly arranged, e.g., to provide greater granularity at the tails of the distribution, where there may be greater information or greater sensitivity to changes in stress associated with variations in the measured heart rate and/or HRRR. Thus for example, the HRRR distribution provided by the HRRR prediction model may provide bpm values corresponding to the 99th percentile, the 96th percentile, the 76th percentile, the 50th percentile, the 23rd percentile, the 3rd percentile, and the 1st percentile of expected heart HRRR values. A variety of data structures may be used to characterize the predicted distribution. For example, the HRRR prediction model may output a list of numbers specifying the HRRR threshold for each of the corresponding percentiles in the HRRR distribution.

A second model may also be created in order to classify an activity of the user. In one aspect, this may include a machine learning model trained as a classification model on a target variable using a multi-class label describing user activities. This may include specific activity types such as walking, running, swimming, sitting, etc., or this may include general activity types such as active, sedentary, sleeping, and so forth. This classification model may in general be trained with inputs concerning motion, heart rate, and so forth, including any of the engineered features described above or variants thereof, and may be adapted, e.g., for specific locations of a wearable physiological monitor, specific user history, and so forth. A variety of techniques for classifying activity based on data from a wearable device are known in the art, and may be used to create a suitable machine learning model or other process for classifying an activity as described herein. Some common learning models include support vector machines, random forests, convolutional neural networks, recurrent neural networks, long/short term memory networks, and so forth. Recurrent neural networks, for example, generally work well with continuous, variable input such as time series data, and may be usefully deployed in this context. In another aspect, a probabilistic model or the like may be used to estimate activity probabilities for individual time segments, and then aggregate these into an activity classification for an interval. More generally, the best model choice will depend on various factors such as the size and quality of the dataset, the complexity of the activity types, and the computational resources available.

It will be understood that the resulting models, tools, algorithms, or other resources for stress scoring may be compressed for local deployment on a wearable physiological monitoring device to facilitate local processing of continuous updates without the need for a server. The machine learning model or other components may also or instead be deployed at a server or other remote processing resource, which permits the use of larger, more refined machine learning models, but may introduce latency into the user experience at times when real time information is desired.

According to the foregoing, in one aspect, the method 700 may include creating a first model to generate a probability distribution of expected heart rate reserve ratios. For example, the first model may include a machine learning model trained to generate a probability distribution of expected heart rate reserve ratios based on a first set of features of training data for a population of users of a type of physiological monitor. In one aspect, generating the probability distribution includes generating a set of heart rate reserve ratios including a threshold heart rate reserve ratio for each of a number of quantiles for the probability distribution. The method 700 may also include creating a second model to identify an activity type. This may include a classification model trained to identify an activity type based on a second set of features of the training data for the population of users of the physiological monitor. These models may generally be used as described herein to create a stress score and provide corresponding coaching or recommendations. In an aspect, the second model is used to modify a stress score to better match an expected stress score of the wearer—e.g., when relatively rigorous activity is identified by the second model.

As shown in step 704, the method 700 may include receiving data for use in a calculation of a stress score. This may, for example, include receiving data from a monitor such as any of the wearable physiological monitors described herein. The monitor may provide data such as heart rate data, motion data, and any other raw or processed data available from the monitor, and any sensor or combination of sensors in the monitor. Receiving data may also or instead include receiving data from a remote data store such as a server that cooperates with the wearable monitor to provide data and analytics to a user. For example, useful summary data such as a resting heart rate for the user, a maximum heart rate for the user, and so forth, may be calculated and stored by the server, and used in calculating a stress score as described herein.

As shown in step 706, the method 700 may include calculating a current heart rate reserve ratio (HRRR), e.g., using Eq. 2 above. In general, the descriptive features (e.g., resting heart rate, baseline resting heart rate, maximum heart rate) may be calculated once and used throughout a period of interest such as a day, two days, a week, or any other interval during which maximum and resting heart rate are expected to remain relatively stable. The current heart rate, however, will typically be a current measurement for use in evaluating the current stress level for a user. For example, this may include an instantaneous measurement of current heart rate. However, because this metric is highly variable, the instantaneous heart rate may advantageously be windowed, averaged, filtered, or otherwise processed to obtain a more consistent measure of current exertion. For example, the current heart rate may be a moving average of the instantaneous heart rate sampled over a recent interval such as one minute, five minutes, or the like. In general, the current HRRR provides a benchmark for comparison to the HRRR distribution predicted by the HRRR prediction model.

As shown in step 708, the method 700 may include predicting an HRRR distribution, e.g., by generating a probability distribution for the HRRR based on current data from a monitor. As described above, this probability distribution (also referred to herein simply as the “HRRR distribution”) provides a range and distribution of expected HRR ratios for a user based on a current user context, as objectively determined based on data such as motion data from a physiological monitor worn by the user.

As shown in step 710, the method 700 may include calculating a stress score, such as an initial stress score for the wearer based on a comparison of the (current) heart rate reserve ratio of the user, as calculated in step 706, to the HRRR distribution generated in step 708. In one aspect, the stress score may be calculated by scoring the current HRRR based on a quantile within the HRRR distribution. For example, the HRRR distribution may be scaled from, e.g., 0 to 1 (or any other suitable numerical range), where zero represents the lowest HRRR in the HRRR distribution, 0.5 represents the median or mean HRRR in the HRRR distribution, and 1 represents the highest HRRR in the HRRR distribution. The interstitial scoring within these intervals may be based on the quantile, within the HRRR distribution, of a current HRRR value. For example, using a linear scaling of quantiles, a current HRRR falling at the 10th percentile would receive a score of 0.1 and a current HRRR falling at the 90th percentile would receive a score of 0.9. As noted above, the HRRR distribution may be generated as a discrete set of quantiles within the distribution, and the initial scoring may also or instead include interpolating the quantiles or otherwise processing the HRRR distribution data and the current HRRR to identify a quantile corresponding to the current HRRR, upon which a score may be based.

Once a quantile has been identified, the reported score may be calculated using any suitable formula. For example, the score may be expressed as a simple scaled version or the quantile (e.g., score=quantile/100). However, other scaling or scoring techniques may also or instead be used to score a current HRRR along the distribution, such as a log scoring or exponential scoring, e.g., in order to weight scoring toward or away from the distribution mean as desired.

While HRRR provides a useful metric for evaluating stress, other metrics are also correlated to stress, and may be used with the techniques described herein to evaluate stress using a probability distribution. For example, instead of HRRR, the method 700 may use heart rate, heart rate variability, respiratory rate, blood pressure, and/or any other cardiac metric. More generally, any physiological metric that can be measured or inferred, and that can be correlated to stress, may be used as a basis for real time stress detection using the techniques described herein, e.g., by creating a model to estimate expected ranges that are correlated to other observable metrics or model inputs, measuring a current value for the corresponding parameter, and then comparing the current value to the estimated range(s).

For example, the method 700 may include calculating and applying a heart rate metric such as one or more of a heart rate reserve, a heart rate reserve ratio, a heart rate variability, an instantaneous heart rate, an aggregate heart rate (e.g., average heart rate), and so on. The method 700 may also or instead include calculating and applying other physiological metrics such as a skin temperature, a core body temperature, a respiratory rate, a skin conductance, a blood pressure, and the like. Similarly, a machine learning model may be trained to generate a probability distribution of corresponding expected heart rate metrics (or other physiological, stress-related metric(s)), which can include any one or more of the aforementioned metrics. It will be further understood that, although any one of a variety of heart rate metrics may be used, it will generally be advantageous to calculate and/or measure a current value for the same one or more metrics that were used to train the machine learning model or other model that generates probability distributions. Increased accuracy may also be achieved under some conditions by combining one or more such metrics, combining the results of models based on such metrics, and/or using a probabilistic approach to select the metric or model that is likely to be most accurate based on one or more features of the one or more metrics.

As shown in step 712, the method 700 may include classifying an activity of the user. This may include applying data from a monitor, e.g., motion data and the like acquired in step 704, to a classification model such as any of those described herein to identify an activity type. More generally, any technique or combination of techniques suitable for determining an activity of the user may be applied to classify the activity, including machine learning techniques, probabilistic techniques, explicit self-reporting (e.g., receiving user input specifying an activity and/or time interval), and so forth.

As shown in step 714, the method 700 may include refining the stress score. In one aspect, this may include refining the stress score based on the activity. For example, the heart rate score can be adjusted based on a predicted probability that a user is sleeping, active, sedentary, and so forth. This may include an aggregated adjustment according to the probability of each type of activity, or a single adjustment based on a most likely predicted activity, or some combination of these. In one aspect, a stress score may be lowered in proportion to a probability that a user is sleeping, where sleep cycles may cause intermittent increases in heart rate that are unrelated to stress. In another aspect, the stress score may be regularized according to probable physical activities. For example, the stress score may be brought closer to an average in proportion to a probability that a user is active, or increased in proportion to a probability that the user is inactive.

Refining the stress score may also or instead include smoothing the stress score, e.g., by averaging a recent history of stress scores, low pass filtering a time series of stress scores over a recent interval, or otherwise adjusting a current stress score, as reported to the user, in order to reduce variability in the instantaneous, calculated value for the stress score. In one aspect, both the instantaneous (or unsmoothed) stress score and a smoothed stress score may be presented to the user.

As shown in step 716, the method 700 may include taking other actions. In general, once a stress score has been determined for a user, the method 700 may include any suitable further processing, e.g. by displaying a stress score, providing recommendations, updating other user metrics, and so forth, all as described herein.

Thus, in one aspect, there is disclosed herein a method including: creating a first model, the first model including a machine learning model trained to generate a probability distribution for a physiological metric based on a first set of features of training data for a population of users of a type of physiological monitor; creating a second model, the second model including a classification model trained to identify an activity type based on a second set of features of the training data for the population of users of the type of physiological monitor; receiving user data from a wearer of a first physiological monitor of the type of physiological monitor; calculating a value for the physiological metric for the wearer based on the user data from the first physiological monitor; generating the probability distribution for the physiological metric for the wearer based on the first set of features of the user data; and calculating a stress score for the wearer based on a comparison of the value for the physiological metric to the probability distribution for the physiological metric. The physiological metric may include a heart rate metric. The physiological metric may include a metric correlated to stress. The physiological metric may include one or more of a heart rate reserve, a heart rate reserve ratio, a heart rate variability, an instantaneous heart rate, an aggregate heart rate (e.g., average heart rate), a skin temperature, a core body temperature, a respiratory rate, a blood pressure, and a skin conductance.

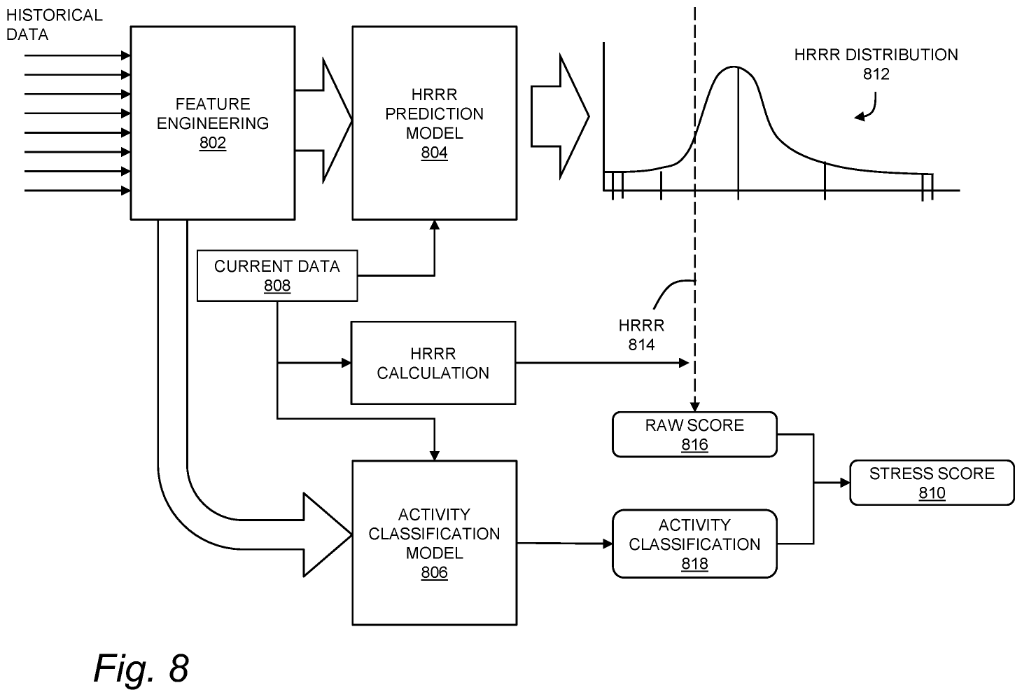

FIG. 8 illustrates a process for calculating a stress score. This may, for example, include use of any of the systems or methods described herein.

In general, feature engineering 802 may be employed to provide feature data based on historical data from a wearable physiological monitor such as motion data, heart rate data, and the like, e.g., for a population of users or for an individual user, or some combination of these. The resulting, engineered features (e.g., summary statistics, moving averages, filtered outputs, and so forth), can provide training data for models such as an HRRR prediction model 804 and a classification model 806, that can be used in turn to support stress scoring as described herein.

Once these models have been created, current data 808 can be used to generate a current stress score 810. For example, engineered features of current data 808 from a wearable physiological monitor, corresponding to the engineered features used to train the HRRR prediction model 804, may be used to generate an HRRR distribution 812 based on a user's current state. A current heart rate for the user may be used along with other user metrics such as (baseline) resting heart rate and maximum heart rate to calculate a current heart rate reserve ratio 814 for the user. As further described herein, this current heart rate reserve ratio 814 may be compared to the HRRR distribution 812 from the HRRR prediction model 804 to identify a quantile for the current HRRR in the context of the user's current activity state. This quantile may be used to create a raw score 816, which may be further refined based on an activity classification 818 (determined, e.g., by applying the activity classification model 806 to the current data 808) to provide the stress score 810 for the user based on the current heart rate reserve ratio 814. This stress score 810 may be further scaled, refined, smoothed, and otherwise post-processed to provide a current stress score suitable for reporting to the user, and/or for making coaching recommendations as described herein.

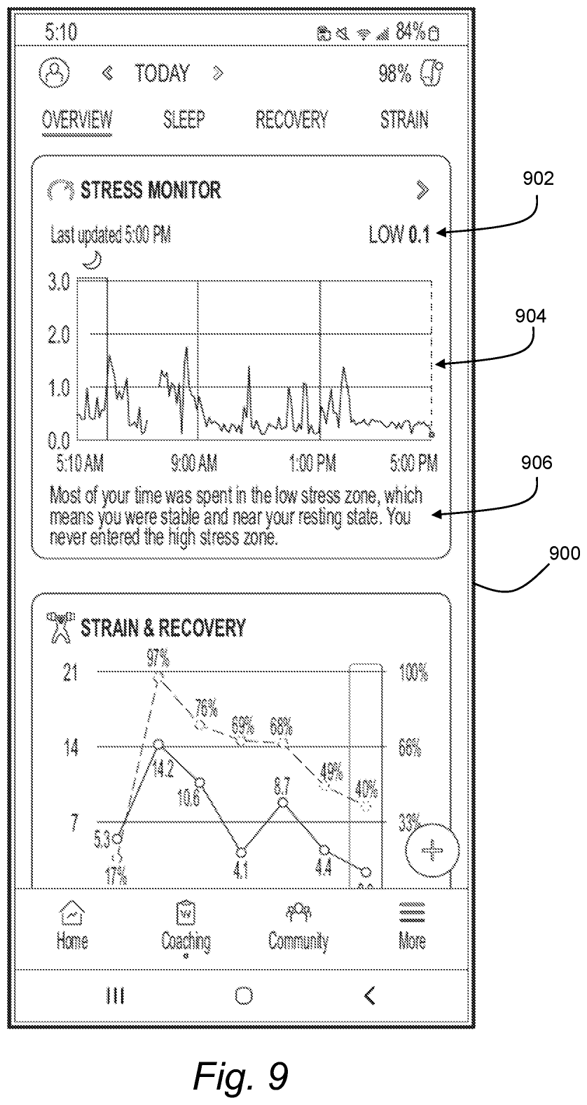

FIG. 9 shows a user interface displaying a dynamic stress score. The user interface 900 may be rendered, for example, on a smartphone or other computing device for a user. In general, a dynamic stress score may be displayed in the user interface 900 in any number of suitable formats. For example, the user interface 900 may display a current value 902 for the dynamic stress score, an historical timeline 904 for the dynamic stress score, one or more coaching recommendations 906 and so forth, along with any other user information requested by or of potential interest to the user. In general, the user interface 900 may also include interactive controls for user interactions with a website, app, or other computing platform supporting the acquisition, analysis, and display of stress data for the user.

### Dynamic Stress Scoring Using Composite Techniques

A dynamic stress score calculation may usefully include combinations of the methods or individual steps described herein. In one aspect, this may include combining different stress calculation techniques to provide an averaged or ensemble measure of stress. For example, the present teachings may include a combination of any of the steps from one or more of the system 500 of FIG. 5, the method 600 of FIG. 6, and the method 700 of FIG. 7.

In another aspect, this may include using different calculation techniques to isolate and report different stress contributors. For example, a calculation that includes motion contributions may be used to estimate stress due to physical activity, while a calculation that varies according to whether the user's heart rate is at or near a resting heart rate, may be used to estimate stress due to psychological stressors. Or a calculation that specifically seeks to remove or mitigate contributions due to physical motion may be used to isolate and estimate stress due to emotional, intellectual, and/or other non-physical stressors. These techniques may be used together to estimate how much of a reported stress score is likely attributable to physical versus non-physical stressors. These techniques may also or instead be used together in order to report separate scores for psychological and physical stressors, which advantageously permits the isolation of physical and psychological stressors, and facilitates the concurrent reporting of both metrics to a user so that the user can evaluate the significance of these measurements together and in combination. Coaching recommendations may also be adapted to indicate what factors appear to be contributing to a current stress score, and to provide suitable interventions where appropriate. For example, where a user is awake and motionless, but exhibiting an elevated heart rate, this may indicate substantial psychological stress. Suitable interventions such as breathwork or meditation may be recommended to alleviate this detected non-physical source of stress. Conversely, where a user has an elevated heart rate during a physical activity such as running, no immediate intervention might be appropriate, particularly where the elevated heart rate is typical of similar levels of physical activity by the user.

In another aspect, a decomposition of stress contributors, along with other user data concerning activity types, sleep activity, exercise, diet, and so forth, can facilitate more customized coaching recommendations concerning lifestyle, work habits, training, and so forth.

More generally, the methods and systems described herein may be used alone or in combination, and any such combinations suitable for dynamic stress scoring, or otherwise evaluating a user's current stress state, are intended to fall within the scope of this disclosure.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

1. A computer program product comprising computer executable code embodied in a non-transitory computer readable medium that, when executing on one or more computing devices, performs the steps of:
creating a first model, the first model including a machine learning model trained to generate a probability distribution of expected heart rate reserve ratios based on a first set of features of training data for a population of users of a type of physiological monitor;
receiving user data from a wearer of a first physiological monitor of the type of physiological monitor;
calculating a heart rate reserve ratio for the wearer based on the user data from the first physiological monitor;
generating the probability distribution of expected heart rate reserve ratios for the wearer based on the first set of features of the user data; and
calculating a stress score for the wearer based on a comparison of the heart rate reserve ratio to the probability distribution of expected heart rate reserve ratios.

2. The computer program product of claim 1, further comprising code that performs the step of creating a second model, the second model including a classification model trained to identify an activity type based on a second set of features of the training data for the population of users of the type of physiological monitor.

3. The computer program product of claim 2, wherein the second model is used to modify the stress score to better match an expected stress score of the wearer.

4. The computer program product of claim 2, wherein the activity type includes one or more of active, sedentary, and sleeping.

5. The computer program product of claim 1, further comprising code that performs the step of classifying an activity type of a user based on a second set of features of the user data and refining the stress score based on the activity type, thereby providing a refined stress score.

6. The computer program product of claim 5, further comprising code that performs the step of providing recommendations to the user based on the refined stress score.

7. The computer program product of claim 1, wherein generating the probability distribution includes generating a set of heart rate reserve ratios corresponding to each of a number of quantiles for the probability distribution.

8. The computer program product of claim 1, further comprising code that performs the step of displaying the stress score on one or more of a wearable monitor and a user device.

9. The computer program product of claim 1, further comprising code that performs the step of generating an intervention recommendation for the wearer based on the stress score.

10. The computer program product of claim 9, wherein the intervention recommendation includes a real time recommendation based on at least one of a current stress score and a current activity.

11. The computer program product of claim 1, further comprising code that performs the step of identifying a threshold for the stress score that is indicative of acute stress.

12. The computer program product of claim 11, further comprising code that performs the step of at least one of reporting the acute stress to the wearer and recommending a remediation for the acute stress.

13. The computer program product of claim 1, further comprising code that performs the step of identifying a threshold for the stress score that is indicative of autonomic activation.

14. A method comprising:
creating a first model, the first model including a machine learning model trained to generate a probability distribution for a physiological metric based on a first set of features of training data for a population of users of a type of physiological monitor;
receiving user data from a wearer of a first physiological monitor of the type of physiological monitor;
calculating a value for the physiological metric for the wearer based on the user data from the first physiological monitor;
generating the probability distribution for the physiological metric for the wearer based on the first set of features of the user data; and
calculating a stress score for the wearer based on a comparison of the value for the physiological metric to the probability distribution for the physiological metric.

15. The method of claim 14, further comprising creating a second model, the second model including a classification model trained to identify an activity type based on a second set of features of the training data for the population of users of the type of physiological monitor.

16. The method of claim 15, wherein the second model is used to modify the stress score to better match an expected stress score of the wearer.

17. The method of claim 14, wherein the physiological metric includes a heart rate metric.

18. The method of claim 14, wherein the physiological metric includes a metric correlated to stress.

19. The method of claim 14, wherein the physiological metric includes one or more of a heart rate reserve, a heart rate reserve ratio, a heart rate variability, an instantaneous heart rate, an aggregate heart rate, a skin temperature, a core body temperature, a respiratory rate, blood pressure, and a skin conductance.

20. A system comprising:
a wearable physiological monitor including one or more sensors and a first processor configured to continuously acquire user data including heart rate data for a wearer based on a signal from the one or more sensors;
a datastore storing a first model, the first model including a machine learning model trained to generate a probability distribution for a physiological metric based on a first set of features of training data for a population of users of a type of physiological monitor;
one or more processors coupled in a communicating relationship with the wearable physiological monitor, the one or more processors configured by computer executable code to receive data from the wearable physiological monitor and to perform the steps of:
receiving the user data from the wearable physiological monitor,
calculating a value for the physiological metric for the wearer based on the user data from the wearable physiological monitor,
generating the probability distribution for the physiological metric for the wearer based on the first set of features of the user data, and
calculating a stress score for the wearer based on a comparison of the value for the physiological metric to the probability distribution for the physiological metric; and
a display device in communication with the one or more processors, the display device including a user interface configured to present a value to the user indicative of the stress score.
